(19) United States
(12) Patent Application Publication
(10) Pub. No.: US 2003/0113776 A1
(43) Pub. Date: Jun. 19, 2003

(54) Methods and compositions for the treatment and diagnosis of cellular proliferation disorders using 54394

(75) Inventor: Mark Williamson, Saugus, MA (US)

Correspondence Address: Steven A. Bossone, Millennium Pharmaceuticals, Inc., 75 Sidney Street, Cambridge, MA, 02139, US

(73) Assignee: Millennium Pharmaceuticals, Inc.

(21) Appl. No.: 10/283,663

(22) Filed: Oct. 30, 2002

Related U.S. Application Data
(60) Provisional application No. 60/335,076, filed on Oct. 31, 2001.

(51) Int. Cl. A61K 48/00; A61K 38/17; A61K 31/00; C12Q 1/68; G01N 33/567; A61K 39/395

(52) U.S. Cl. 435/6; 435/7.21; 514/44; 424/146.1; 514/1; 514/2

(57) ABSTRACT

The present invention relates to methods and compositions for the treatment and diagnosis of cellular proliferation disorders, including, but not limited to, breast cancer, ovarian cancer, lung cancer, and colon cancer. The invention further provides methods for identifying a compound capable of treating a cellular proliferation disorders disorder or modulating cellular proliferation. The invention also provides a method for modulating cellular proliferation, e.g., modulating cellular proliferation in a subject. In addition, the invention provides a method for treating a subject having a cellular proliferation disorder characterized by aberrant 54394 polypeptide activity or aberrant 54394 nucleic acid expression.

This application claims priority to U.S. provisional application No. 60/335,076, filed Oct. 31, 2001, the entire contents of which are incorporated herein by reference.

Colorectal cancer is the fourth most common cancer worldwide and the second most common cause of cancer deaths. Within the United States alone, there will be over 150,000 new cases and 55,000 deaths this year. In fact, it is postulated that 50% of the Western population will develop a colorectal tumor by the age of 70, with 10% of these tumors progressing to malignancy. Despite advances in therapeutic treatment, the prognosis remains poor, with only a five-year survival rate around 45%. Although the progression of the disease has been well characterized (areas of dysplasia within the colon develop into polyps, which eventually have the potential to become adenocarcinomas; adenocarcinomas become invasive and metastasize to various regions of the body, predominately the liver), diagnosis is primarily made during later stages of the disease.

Lung cancer is among the most common cancers in the Western world. In the United States, there were approximately 170,000 new cases of lung cancer in 1999. Since the mid-1990s, about 150,000 Americans have died each year from this disease. Lung cancer is the leading category of cancer death in men, and—since the late 1980s—it has surpassed breast cancer as the leading category of cancer death in women. Findings from the U.S. National Cancer Institute (NCI) indicate that the upward trend in cancer-related death is due to the rapidly increasing rate of lung cancer mortality. Statistical projections suggest that lung cancer mortality in this decade will continue to rise to a rate of over 50 deaths per year per 100,000 population in America. Current lung cancer prevention programs are not expected to influence lung cancer death rates until after the year 2000. There is a close relationship between the number of lung cancer cases and lung cancer deaths in America. This is because of the low 5-year survival rate for this disease. Although lung cancer survival rates have improved over the last 40 years, the percentage (approximately 13%) continues to be low in comparison to other cancers.

Given the prevalence of these disorders, and the lack of effective cures and early diagnostics, there currently exists a great need for methods and compositions which can serve as markers before the onset of symptoms and which can serve as a means for identifying therapeutics to treat and or cure these disorders.

The present invention provides methods and compositions for the diagnosis and treatment of cellular proliferation disorders, e.g., colon cancer, lung cancer, and/or breast cancer. The present invention is based, at least in part, on the discovery that expression of the 54394 gene (a lysophospholipase) is upregulated in tumors (e.g., lung and colon tumors). The present invention is further based, at least in part, on the discovery that 54394 expression is regulated during the cell cycle, and that 54394 expression is downregulated in HCT116 k-ras disrupted colon cancer cells. The invention is still further based, at least in part, on the discovery that 54394 may be involved in the regulation of the levels of lysophospholipids in cells and in the regulation of cellular signaling and adhesion. Without intending to be limited by mechanism, it is believed that modulation, e.g., inhibition, of 54394 activity may modulate the levels of lysophospholipids in the cell and may therefore modulate, e.g., inhibit, cellular proliferation and promote apoptosis.

Accordingly, the present invention provides methods for the diagnosis and treatment of cellular proliferation disorders including, but not limited to, cancer, e.g., breast cancer, ovarian cancer, lung cancer, and colon cancer.

In one aspect, the invention provides methods for identifying a compound capable of treating a cellular proliferation disorder, e.g., breast cancer, ovarian cancer, lung cancer, and colon cancer. The method includes assaying the ability of the compound to modulate 54394 nucleic acid expression or 54394 polypeptide activity. In one embodiment, the ability of the compound to modulate nucleic acid expression or 54394 polypeptide activity is determined by detecting the lysophospholipase activity of a cell. In another embodiment, the ability of the compound to modulate nucleic acid expression or 54394 polypeptide activity is determined by detecting modulation of cellular proliferation in a cell.

In another aspect, the invention provides methods for identifying a compound capable of modulating cellular proliferation. The method includes contacting a cell expressing a 54394 nucleic acid or polypeptide (e.g., a breast cell, a breast tumor cell, an ovary cell, an ovarian tumor cell, a lung cell, a lung tumor cell, a colon cell, and/or a colon tumor cell) with a test compound and assaying the ability of the test compound to modulate the expression of a 54394 nucleic acid or the activity of a 54394 polypeptide.

In a further aspect, the invention features a method for modulating cellular proliferation. The method includes contacting a cell (e.g., a breast cell, a breast tumor cell, an ovary cell, an ovarian tumor cell, a lung cell, a lung tumor cell, a colon cell, and/or a colon tumor cell) with a 54394 modulator, for example, an anti-54394 antibody, a 54394 polypeptide comprising the amino acid sequence of SEQ ID NO:2, or a fragment thereof, a 54394 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, a small molecule, an antisense 54394 nucleic acid molecule, a nucleic acid molecule of SEQ ID NO:1, or a fragment thereof, or a ribozyme.

In yet another aspect, the invention features a method for treating a subject having a cellular proliferation disorder, e.g., a cellular proliferation disorder characterized by aberrant 54394 polypeptide activity or aberrant 54394 nucleic acid expression, such as breast cancer, ovarian cancer, lung cancer, and colon cancer. The method includes administering to the subject a therapeutically effective amount of a 54394 modulator, e.g., in a pharmaceutically acceptable formulation or by using a gene therapy vector. In one embodiment, the 54394 modulator may be a small molecule, an anti-54394 antibody, a 54394 polypeptide comprising the amino acid sequence of SEQ ID NO:2, or a fragment thereof, a 54394 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, an antisense 54394 nucleic acid molecule, a nucleic acid molecule of SEQ ID NO:1, or a fragment thereof, or a ribozyme.

In another aspect, the invention provides a method for modulating, e.g., increasing or decreasing, cellular proliferation in a subject by administering to the subject a 54394 modulator.

Other features and advantages of the invention will be apparent from the following detailed description and claims.

Table 1 depicts the expression levels of human 54394 mRNA in various human cell types and tissues, as determined by Taqman analysis. Sample No.: (1) normal artery; (2) diseased aorta; (3) normal vein; (4) coronary smooth muscle cells; (5) human umbilical vein endothelial cells (HUVECs); (6) hemangioma; (7) normal heart; (8) heart (congestive heart failure); (9) kidney; (10) skeletal muscle; (11) normal adipose tissue; (12) pancreas; (13) differentiated osteoclasts; (14) normal skin; (15) normal spinal cord; (16) normal brain cortex; (17) normal brain hypothalamus; (18) nerve; (19) dorsal root ganglion; (20) normal breast; (21) breast tumor; (22) normal ovary; (23) ovarian tumor; (24) normal prostate; (25) salivary gland; (26) normal colon; (27) colon tumor; (28) normal lung; (29) lung tumor; (30) lung (chronic obstructive pulmonary disease); (31) colon (inflammatory bowel disease); (32) normal liver; (33) liver fibrosis; (34) normal spleen; (35) normal tonsil; (36) normal lymph node; (37) normal small intestine; (38) macrophages; (39) synovium; (40) activated peripheral blood mononuclear cells; (41) neutrophils; (42) megakaryocytes; (43) erythroid cells; (44) positive control.

Table 2 depicts the expression levels of human 54394 mRNA in various human tumors, as determined by Taqman analysis. Sample No.: (1-2) normal breast; (3) breast tumor (MD-IDC); (4) breast tumor; (5) breast tumor (PD-); (6) breast tumor (IDC); (7) breast tumor (ILC (LG)); (8) lymph; (9) lung (breast metastasis); (10-1 1) normal ovary; (12-16) ovary tumor; (17-19) normal lung; (20) lung tumor (SmC); (21) lung tumor (PDNSCC); (22) lung tumor (SCC); (23-24) lung tumor (ACA); (25-27) normal colon; (28-29) colon tumor (MD); (30) colon tumor; (31-32) colon tumor—liver metastasis; (33) normal liver (female); (34-35) cervix—squamous cell carcinoma; (36) human microvascular endothelial cells (HMVECs)—arrested; (37) human microvascular endothelial cells (HMVECs)—proliferating; (38) hemangioma; (39) HCT116—normoxic; (40) HCT116—hypoxic; (41-42) normal prostate; (43) prostate tumor—St. 5; (44) prostate tumor—St. 7.

Table 3 depicts the expression levels of human 54394 mRNA in various xenograft (tumorigenic) cell lines, as determined by Taqman analysis. Sample No.: (1) MCF-7 breast tumor; (2) ZR75 breast tumor; (3) T47D breast tumor; (4) MDA 231 breast tumor; (5) MDA 435 breast tumor; (6) SKBr3 breast tumor; (7) DLD 1 colon tumor (stage C); (8) SW480 colon tumor (stage B); (9) SW620 colon tumor (stage C); (10) HCT 116 colon tumor; (11) HT29 colon tumor; (12) Colo 205 colon tumor; (13) NCIH125 lung tumor; (14) NCIH67 lung tumor; (15) NCIH322 lung tumor; (16) NCIH460 lung tumor; (17) A549 lung tumor; (18) normal human bronchial epithelium (NHBE); (19) SKOV-3 ovary tumor; (20) OVCAR-3 ovary tumor; (21) 293 baby kidney cells; (22) 293T baby kidney cells.

Table 4 depicts the expression levels of human 54394 mRNA in various staged colon tumors, as determined by Taqman analysis. Sample No.: (1-5) normal colon; (6) adenomas; (7-11) colonic ACA-B; (12-17) colonic ACA-C; (18-23) normal liver; (24-28) liver metastasis; (29) abdominal metastasis.

Table 5 depicts the expression levels of human 54394 mRNA in various colon metastases, as determined by Taqman analysis. Sample No.: (1-3) normal colon; (4-5) colonic ACA-C; (6) colonic ACA-B; (7) adenocarcinoma; (8-22) colon metastasis to the liver; (23-25) normal liver.

Table 6 depicts the expression levels of human 54394 mRNA in a k-ras disrupted HCT116 colon tumor cell lines, as determined by Taqman analysis. Sample No.: (1) JHCT116 (one activated k-ras allele and one normal k-ras allele); (2) HK2-6 (the normal k-ras allele is disrupted); (3) HKe3 (the activated allele is disrupted); (4) HKh2 (the activated allele is disrupted); (5) e3HAM#9 (HKe3 transfected with activated k-ras).

Table 7 depicts the expression levels of human 54394 mRNA in synchronized tumor cells induced to progress through the cell cycle, as determined by Taqman analysis. Sample No.: (1) HCT116, aphidicolin, t=0; (2) HCT116, aphidicolin, t=3; (3) HCT116, aphidicolin, t=6; (4) HCT116, aphidicolin, t=9; (5) HCT116, aphidicolin, t=12; (6) HCT116, aphidicolin, t=15; (7) HCT116, aphidicolin, t=18; (8) HCT116, aphidicolin, t=21; (9) HCT116, aphidicolin, t=24; (10) HCT116, nocodazole, t=0; (11) HCT116, nocodazole, t=3; (12) HCT116, nocodazole, t=6; (13) HCT116, nocodazole, t=9; (14) HCT116, nocodazole, t=18; (15) HCT116, nocodazole, t=21; (16) HCT116, nocodazole, t=24; (17) DLD, nocodazole, t=3; (18) DLD, nocodazole, t=6; (19) DLD, nocodazole, t=9; (20) DLD, nocodazole, t=12; (21) DLD, nocodazole, t=15; (22) DLD, nocodazole, t=18; (23) A549, mimo, t=0; (24) A549, mimo, t=3; (25) A549, mimo, t=6; (26) A549, mimo, t=9; (27) A549, mimo, t=15; (28) A549, mimo, t=18; (29) A549, mimo, t=21; (30) A549, mimo, t=24; (31) MCF10A, mimo, t=0; (32) MCF10A, mimo, t=3; (33) MCF10A, mimo, t=6; (34) MCF10A, mimo, t=9; (35) MCF10A, mimo, t=12; (36) MCF10A, mimo, t=18; (37) MCF10A, mimo, t=21; (38) MCF10A, mimo, t=24.

Table 8 depicts the expression levels of human 54394 mRNA in various in vitro oncogene cell models, as determined by Taqman analysis. Sample No.: (1) SMAD4-SW480 control; (2) SMAD4-SW480 24 hours; (3) SMAD4-SW480 48 hours; (4) SMAD4-SW480 72 hours; (5) L51747 mucinous; (6) HT29 non-mucinous; (7) SW620 non-mucinous; (8) CSC-1 normal; (9) NCM-460 normal; (10) HCT116 RER+; (11) SW480 RER−/−; (12) CACO RER−/−; (13) JHCT116; (14) DKO1; (15) DKO4; (16) DKS-8; (17) Hke3; (18) HKh2; (19) HK2-6; (20) e3Ham#9; (21) APC5−/−; (22) APC6−/−; (23) APC1+/+; (24) APC13+/+.

Table 9 depicts the expression levels of human 54394 mRNA in various in vitro oncogene cell models, as determined by Taqman analysis. Sample No.: (1) SMAD4-SW480 24 hours; (2) SMAD4-SW480 48 hours; (3) SMAD4-SW480 72 hours; (4) L51747 mucinous; (5) HT29 non-mucinous; (6) SW620 non-mucinous; (7) CSC-1 normal; (8) NCM-460 normal; (9) HCT116 RER+; (10) SW48 RER+; (11) SW480 RER−/−; (12) CACO RER−/−; (13) JDLD-1; (14) JHCT116; (15) DKO1; (16) DKO4; (17) DKS-8; (18) Hke3; (19) HKh2; (20) HK2-6; (21) e3Ham#9.

Table 10 depicts the expression levels of human 54394 mRNA in various in vitro oncogene cell models, as determined by Taqman analysis. Sample No.: (1) SMAD4-SW480 24 hours; (2) SMAD4-SW480 48 hours; (3) SMAD4-SW480 72 hours; (4) L51747 mucinous; (5) HT29 non-mucinous; (6) SW620 non-mucinous; (7) CSC-1 normal; (8) NCM-460 normal; (9) HCT116 RER+; (10) SW48 RER+; (11) SW480 RER−/−; (12) CACO RER−/−; (13) JDLD-1; (14) JHCT116; (15) DKO1; (16) DKO4; (17) DKS-8; (18) Hke3; (19) HKh2; (20) HK2-6; (21) e3Ham#9.

TABLE 1

| Sample | | Relative |
|---|---|---|
| Number | Tissue Type | Expression |
| 1 | Artery normal | 41.2346 |
| 2 | Aorta diseased | 52.7385 |
| 3 | Vein normal | 47.5306 |
| 4 | Coronary SMC | 58.517 |
| 5 | HUVEC | 41.8102 |
| 6 | Hemangioma | 35.2801 |
| 7 | Heart normal | 47.2023 |
| 8 | Heart CHF | 63.1532 |
| 9 | Kidney | 47.8612 |
| 10 | Skeletal Muscle | 31.5766 |
| 11 | Adipose normal | 6.8485 |
| 12 | Pancreas | 19.7774 |
| 13 | Osteoclasts (diff) | 0 |
| 14 | Skin normal | 1.1775 |
| 15 | Spinal cord normal | 0.8183 |
| 16 | Brain Cortex normal | 28.9559 |
| 17 | Brain Hypothalamus normal | 13.8882 |
| 18 | Nerve | 0 |
| 19 | DRG (Dorsal Root Ganglion) | 0.0341 |
| 20 | Breast normal | 0 |
| 21 | Breast tumor | 0 |
| 22 | Ovary normal | 0 |
| 23 | Ovary Tumor | 0 |
| 24 | Prostate Normal | 0 |
| 25 | Salivary glands | 0 |
| 26 | Colon normal | 0 |
| 27 | Colon Tumor | 0 |
| 28 | Lung normal | 0 |
| 29 | Lung tumor | 0.059 |
| 30 | Lung COPD | 0 |
| 31 | Colon IBD | 0 |
| 32 | Liver normal | 0 |
| 33 | Liver fibrosis | 0 |
| 34 | Spleen normal | 0 |
| 35 | Tonsil normal | 0 |
| 36 | Lymph node normal | 0 |
| 37 | Small intestine normal | 0 |
| 38 | Macrophages | 0 |
| 39 | Synovium | 0 |
| 40 | Activated PBMC | 0 |
| 41 | Neutrophils | 0.4782 |
| 42 | Megakaryocytes | 19.7774 |
| 43 | Erythroid | 30.9268 |
| 44 | positive control | 49.549 |

TABLE 2

| Sample | | Relative |
|---|---|---|
| Number | Tissue Type | Expression |
| 1 | ONC 038 Breast N | 0.00 |
| 2 | CHT 1228 Breast N | 0.00 |
| 3 | MDA 304 Breast T: MD-IDC | 0.00 |
| 4 | CHT 2002 Breast T: IDC | 0.00 |
| 5 | MDA 236-Breast T:PD- | 0.00 |
| | IDC(ILC?) | |
| 6 | CHT 562 Breast T: IDC | 0.00 |
| 7 | NDR 138 Breast T ILC (LG) | 0.00 |
| 8 | CHT 1841 Lymph node | 0.00 |
| | (Breast met) | |
| 9 | PIT 58 Lung (Breast met) | 0.00 |
| 10 | CHT 620 Ovary N | 0.00 |
| 11 | CHT 619 Ovary N | 0.00 |
| 12 | CLN 012 Ovary T | 7.19 |
| 13 | CLN 07 Ovary T | 0.00 |
| 14 | CLN 17 Ovary T | 0.00 |
| 15 | MDA 25 Ovary T | 0.00 |
| 16 | CLN 08 Ovary T | 0.00 |
| 17 | PIT 298 Lung N | 0.00 |
| 18 | MDA 185 Lung N | 0.00 |
| 19 | CLN 930 Lung N | 0.00 |
| 20 | MPI 215 Lung T-SmC | 0.00 |
| 21 | CHT 832 Lung T-PDNSCCL | 0.00 |
| 22 | MDA 262 Lung T-SCC | 80.49 |
| 23 | CHT 793 Lung T-ACA | 14.03 |
| 24 | CHT 331 Lung T-ACA | 27.87 |
| 25 | CHT 405 Colon N | 0.00 |
| 26 | CHT 1685 Colon N | 0.00 |
| 27 | CHT 371 Colon N | 0.55 |
| 28 | CHT 382 Colon T: MD | 99.44 |
| 29 | CHT 528 Colon T: MD | 44.66 |
| 30 | CLN 609 Colon T | 0.18 |
| 31 | CHT 340 Colon-Liver Met | 0.00 |
| 32 | CHT 1637 Colon-Liver Met | 0.00 |
| 33 | PIT 260 Liver N (female) | 0.00 |
| 34 | CHT 1653 Cervix Squamous CC | 36.65 |
| 35 | CHT 569 Cervix Squamous CC | 0.00 |
| 36 | A24 HMVEC-Arr | 0.00 |
| 37 | C48 HMVEC-Prol | 0.00 |
| 38 | Pooled Hemangiomas | 0.00 |
| 39 | HCT116N22 Normoxic | 47.70 |
| 40 | HCT116H22 Hypoxic | 4.00 |
| 41 | CHT 31 Prostate N | 0.54 |
| 42 | CHT 33 Prostate N | 0.00 |
| 43 | CHT 1269 Prostate T: St 5 | 0.00 |
| 44 | PIT 120 Prostate T: St 7 | 0.00 |

TABLE 3

| Sample | | Relative |
|---|---|---|
| Number | Tissue Type | Expression |
| 1 | MCF-7 Breast T | 6.71 |
| 2 | ZR75 Breast T | 17.28 |
| 3 | T47D Breast T | 18.33 |
| 4 | MDA 231 Breast T | 0.05 |
| 5 | MDA 435 Breast T | 0.00 |
| 6 | SKBr3 Breast | 0.00 |
| 7 | DLD 1 Colon T (stageC) | 257.03 |
| 8 | SW480 Colon T (stage B) | 0.00 |
| 9 | SW620 Colon T (stageC) | 3.13 |
| 10 | HCT116 | 1.89 |
| 11 | HT29 | 0.00 |
| 12 | Colo 205 | 0.00 |
| 13 | NCIH125 | 0.70 |
| 14 | NCIH67 | 3.68 |
| 15 | NCIH322 | 2.54 |
| 16 | NCIH460 | 8.85 |
| 17 | A549 | 2.96 |
| 18 | NHBE | 1.04 |
| 19 | SKOV-3 ovary | 0.00 |
| 20 | OVCAR-3 ovary | 0.00 |
| 21 | 293 Baby Kidney | 27.20 |
| 22 | 293T Baby Kidney | 132.13 |

TABLE 4

| Sample | | Relative |
|---|---|---|
| Number | Tissue Type | Expression |
| 1 | CHT 2232 Colon N | 1.39 |
| 2 | CHT 410 Colon N | 0.00 |
| 3 | CHT 425 Colon N | 0.00 |
| 4 | PIT 281 Colon N | 3.84 |
| 5 | NDR 211 Colon N | 0.00 |
| 6 | CHT 887 Adenomas | 0.37 |
| 7 | CHT 414 Colonic ACA-B | 0.00 |
| 8 | CHT 841 Colonic ACA-B | 0.00 |
| 9 | CHT 807 Colonic ACA-B | 0.20 |
| 10 | CHT 382 Colonic ACA-B | 2.04 |
| 11 | CHT 377 Colonic ACA-B | 0.00 |
| 12 | CHT 520 Colonic ACA-C | 0.15 |
| 13 | CHT 596 Colonic ACA-C | 0.00 |
| 14 | CHT 907 Colonic ACA-C | 0.15 |
| 15 | CHT 372 Colonic ACA-C | 3.59 |
| 16 | NDR 210 Colonic ACA-C | 1.29 |
| 17 | CHT 1365 Colonic ACA-C | 2.82 |
| 18 | CLN 740 Liver N | 17.16 |
| 19 | CLN 741 Liver N | 34.92 |
| 20 | NDR 165 Liver N | 4.98 |
| 21 | NDR 150 Liver N | 21.27 |
| 22 | PIT 236 Liver N | 6.94 |
| 23 | CHT 1878 Liver N | 11.72 |
| 24 | CHT 077 Col Liver Met | 16.35 |
| 25 | CHT 119 Col Liver Met | 3.37 |
| 26 | CHT 131 Col Liver Met | 22.64 |
| 27 | CHT 218 Col Liver Met | 23.85 |
| 28 | CHT 755 Col Liver Met | 19.78 |
| 29 | CHT 215 Col Abdominal Met | 1.42 |

TABLE 5

| Sample | | Relative |
|---|---|---|
| Number | Tissue Type | Expression |
| 1 | CHT 371 Colon N | 2.97 |
| 2 | CHT 523 Colon N | 44.97 |
| 3 | NDR 104 Colon N | 11.05 |
| 4 | CHT 520 Colonic ACA-C | 9.82 |
| 5 | CHT 1365 Colonic ACA-C | 78.56 |
| 6 | CHT 382 Colonic ACA-B | 16.35 |
| 7 | CHT 122 Adenocarcinoma | 7.07 |
| 8 | CHT 077 Liver-Colon Mets | 28.36 |
| 9 | CHT 739 Liver-Colon Mets | 1.81 |
| 10 | CHT 755 Liver-Colon Mets | 5.92 |
| 11 | CHT001 Liver-Colon Mets | 0.00 |
| 12 | CHT 084 Liver-Colon Mets | 12.65 |
| 13 | CHT 113 Liver-Colon Mets | 9.92 |
| 14 | CHT 127 Liver-Colon Mets | 23.44 |
| 15 | CHT 218 Liver-Colon Mets | 5.76 |
| 16 | CHT 220 Liver-Colon Mets | 33.38 |
| 17 | CHT 324 Liver-Colon Mets | 6.99 |
| 18 | CHT 340 Liver-Colon Met | 28.56 |
| 19 | CHT 530 Liver-Colon Met | 10.24 |
| 20 | CHT 849 Liver-Colon Met | 9.82 |
| 21 | CHT 1637 Liver-Colon Met | 36.65 |
| 22 | CHT131 Liver-Colon Met | 66.75 |
| 23 | NDR 165 Liver Normal | 20.69 |
| 24 | NDR 150 Liver Normal | 52.37 |
| 25 | PIT 236 Liver Normal | 6.35 |

TABLE 6

| Sample | | Relative |
|---|---|---|
| Number | Tissue Type | Expression |
| 1 | JHCT116 | 73.05 |
| 2 | HK2-6 | 56.92 |
| 3 | HKe3 | 15.30 |
| 4 | HKh2 | 25.38 |
| 5 | e3Ham#9 | 38.47 |

TABLE 7

| Sample | | Relative |
|---|---|---|
| Number | Tissue Type | Expression |
| 1 | HCT 116 Aphidl t = 0 | 0.00 |
| 2 | HCT 116 Aphidl t = 3 | 0.00 |
| 3 | HCT 116 Aphidl t = 6 | 0.00 |
| 4 | HCT 116 Aphidl t = 9 | 0.18 |
| 5 | HCT 116 Aphidl t = 12 | 0.24 |
| 6 | HCT 116 Aphidl t = 15 | 0.13 |
| 7 | HCT 116 Aphidl t = 18 | 0.46 |
| 8 | HCT 116 Aphidl t = 21 | 0.00 |
| 9 | HCT 116 Aphidl t = 24 | 0.00 |
| 10 | HCT 116 Noc t = 0 | 5.05 |
| 11 | HCT 116 Noc t = 3 | 3.27 |
| 12 | HCT 116 Noc t = 6 | 11.60 |
| 13 | HCT 116 Noc t = 9 | 2.19 |
| 14 | HCT 116 Noc t = 18 | 6.82 |
| 15 | HCT 116 Noc t = 21 | 12.87 |
| 16 | HCT 116 Noc t = 24 | 5.51 |
| 17 | DLD noc t = 3 | 95.39 |
| 18 | DLD noc t = 6 | 151.25 |
| 19 | DLD noc t = 9 | 119.08 |
| 20 | DLD noc t = 12 | 144.09 |
| 21 | DLD noc t = 15 | 205.19 |
| 22 | DLD noc t = 18 | 122.43 |
| 23 | A549 Mimo t = 0 | 68.87 |
| 24 | A549 Mimo t = 3 | 85.97 |
| 25 | A549 Mimo t = 6 | 61.00 |
| 26 | A549 Mimo t = 9 | 39.97 |
| 27 | A549 Mimo t = 15 | 27.68 |
| 28 | A549 Mimo t = 18 | 21.49 |
| 29 | A549 Mimo t = 21 | 22.72 |
| 30 | A549 Mimo t = 24 | 32.69 |
| 31 | MCF10A Mimo t = 0 | 2.09 |
| 32 | MCF10A Mimo t = 3 | 3.10 |
| 33 | MCF10A Mimo t = 6 | 0.39 |
| 34 | MCF10A Mimo t = 9 | 0.00 |
| 35 | MCF10A Mimo t = 12 | 0.00 |
| 36 | MCF10A Mimo t = 18 | 1.89 |
| 37 | MCF10A Mimo t = 21 | 0.00 |
| 38 | MCF10A Mimo t = 24 | 0.00 |

TABLE 8

| Sample | | Relative |
|---|---|---|
| Number | Tissue Type | Expression |
| 1 | SMAD4-SW480 C | 5.90 |
| 2 | SMAD4-SW480 24 HR | 29.16 |
| 3 | SMAD4-SW480 48 HR | 20.40 |
| 4 | SMAD4-SW480 72 HR | 29.77 |
| 5 | L51747-MUCINOUS | 34.20 |
| 6 | HT29 NON-MUCINOUS | 5.30 |
| 7 | SW620 NON-MUCINOUS | 2.80 |
| 8 | CSC-1 NORMAL | 43.89 |
| 9 | NCM-460 NORMAL | 28.86 |
| 10 | HCT116 RER+ | 23.20 |
| 11 | SW480 RER−/− | 2.78 |
| 12 | CACO- RER−/− | 18.14 |
| 13 | JHCT116 | 73.05 |
| 14 | DKO1 | 195.47 |
| 15 | DKO4 | 251.74 |
| 16 | DKS-8 | 162.67 |
| 17 | HKe3 | 15.30 |
| 18 | HKh2 | 25.38 |
| 19 | HK2-6 | 56.92 |
| 20 | e3Ham#9 | 38.47 |
| 21 | APC5−/− | 0.00 |
| 22 | APC6−/− | 2.00 |
| 23 | APC1+/+ | 0.31 |
| 24 | APC13+/+ | 0.61 |

TABLE 9

| Sample | | Relative |
|---|---|---|
| Number | Tissue Type | Expression |
| | SMAD4-SW480 24 hr | 21.8 |
| | SMAD4-SW480 48 hr | 55.4 |
| | SMAD4-SW480 72 hr | 60.8 |
| | L51747-Mucinous | 68.9 |
| | HT29 Non-Mucinous | 48.7 |
| | SW620 Non-Mucinous | 11 |
| | CSC-1 Normal | 22.5 |
| | NCM-460 Normal | 42.3 |
| | HCT 116 RER+ | 110.8 |
| | SW48 RER+ | 90.9 |
| | SW480 RER−/− | 10.2 |
| | CACO- RER−/− | 7.4 |
| | JDLD-1 | 71.6 |
| | JHCT116 | 51.3 |
| | DKO1 | 109.3 |
| | DKO4 | 149.8 |
| | DKS-8 | 12.9 |
| | HKe3 | 22 |
| | HKh2 | 33.6 |
| | HK2-6 | 39.2 |
| | e3Ham#9 | 114.3 |

TABLE 10

| Sample | | Relative |
|---|---|---|
| Number | Tissue Type | Expression |
| 1 | SMAD4-SW480 24 hr | 3.15 |
| 2 | SMAD4-SW480 48 hr | 3.28 |
| 3 | SMAD4-SW480 72 hr | 2.88 |
| 4 | L51747-Mucinous | 8.85 |
| 5 | HT29 Non-Mucinous | 4.76 |
| 6 | SW620 Non-Mucinous | 6.41 |
| 7 | CSC-1 Normal | 7.95 |
| 8 | NCM-460 Normal | 7.09 |
| 9 | HCT 116 RER+ | 7.31 |
| 10 | SW48 RER+ | 21.12 |
| 11 | SW480 RER−/− | 4.57 |
| 12 | CACO- RER−/− | 3.27 |
| 13 | JDLD-1 | 118.67 |
| 14 | JHCT116 | 19.64 |
| 15 | DKO1 | 94.08 |
| 16 | DKO4 | 98.07 |
| 17 | DKS-8 | 39.01 |
| 18 | HKe3 | 11.80 |
| 19 | HKh2 | 6.71 |
| 20 | HK2-6 | 19.98 |
| 21 | e3Ham#9 | 14.03 |

The present invention provides methods and compositions for the diagnosis and treatment of cellular proliferation disorders, e.g., colon cancer, lung cancer, and/or breast cancer. The present invention is based, at least in part, on the discovery that expression of the 54394 gene (a lysophospholipase) is upregulated in tumors (e.g., lung and colon tumors). The present invention is further based, at least in part, on the discovery that 54394 expression is regulated during the cell cycle, and that 54394 expression is downregulated in HCT116 k-ras disrupted colon cancer cells. The invention is still further based, at least in part, on the discovery that 54394 may be involved in the regulation of the levels of lysophospholipids in cells and in the regulation of cellular signaling and adhesion. Without intending to be limited by mechanism, it is believed that modulation, e.g., inhibition, of 54394 activity may modulate the levels of lysophospholipids in the cell and may therefore modulate, e.g., inhibit, cellular proliferation and promote apoptosis.

54394 is a member of a class of enzymes called lysophospholipases, which are widely distributed enzymes that hydrolyze phospholipids. Lysophospholipids are important components of cell membranes and are involved in a variety of physiological and pathological processes. The in vivo levels of lysophospholipids are critical for cell survival and function. The accumulation of lysophospholipids can perturb the activities of many membrane-bound signal-transducing enzymes, distort cell membrane integrity, and even cause cell lysis.

54394 also shows similarity to the family of palmitoyl protein thioesterases. These enzymes are involved in the deacylation of palmitoylated proteins. Overexpression of palmitoyl protein thioesterase 1 (PPT1) in neuroblastoma cells protects cells against cell death. Inhibition of PPT1 activity in these cells increases their susceptibility to apoptosis, suggesting that protein palmitoylation may play an important role in the cell survival pathway. Additionally, Ras requires palmitoylation for its biological activity.

The 54394 modulators identified according to the methods of the invention can be used to modulate cellular proliferation (e.g., in breast, lung, and/or colon cells) and are, therefore, useful in treating, diagnosing, or prognosing cellular proliferation disorders. For example, inhibition of the activity of a 54394 molecule can inhibit cellular proliferation, thereby inhibiting tumorigenesis in the subject. Thus, the 54394 modulators identified using the assays described herein can be used to treat cellular proliferation disorders (e.g., cancer) and/or disorders which are secondary to such disorders. Alternatively, 54394 modulators can increase cellular proliferation by increasing 54394 activity in a subject. Thus, 54394 modulators are also useful in the treatment of undesirable cell death, e.g., neurodegenerative disorders.

As used herein, “cellular proliferation disorders” include those disorders that affect cellular proliferation, growth, apoptosis, differentiation, and/or migration processes. As used herein, a “cellular proliferation, growth, apoptosis, differentiation, and/or migration process” is a process by which a cell increases in number, size or content, by which a cell undergoes programmed cell death, by which a cell develops a specialized set of characteristics which differ from that of other cells, or by which a cell moves closer to or further from a particular location or stimulus. Examples of cellular proliferation disorders include cancer, e.g., breast cancer, colon cancer, lung cancer, ovarian cancer, as well as other types of carcinomas, sarcomas, lymphomas, and/or leukemias; tumor angiogenesis and metastasis; skeletal dysplasia; hepatic disorders; and hematopoietic and/or myeloproliferative disorders. Other examples of disorders characterized by aberrant regulation of apoptosis include stroke-associated cell death and neurodegenerative disorders such as Alzheimer's disease, dementias related to Alzheimer's disease (such as Pick's disease), Parkinson's and other Lewy diffuse body diseases, senile dementia, and Huntington's disease.

As used interchangeably herein, “54394 activity,” “biological activity of 54394” or “functional activity of 54394,” includes an activity exerted by a 54394 protein, polypeptide or nucleic acid molecule on a 54394 responsive cell or tissue (e.g., breast, lung, or colon) or on a 54394 protein substrate, as determined in vivo, or in vitro, according to standard techniques. 54394 activity can be a direct activity, such as an association with a 54394-target molecule. As used herein, a “substrate” or “target molecule” or “binding partner” is a molecule with which a 54394 protein binds or interacts in nature, such that 54394-mediated function, e.g., hydrolysis of a lysophospholipid. A 54394 target molecule can be a non-54394 molecule (e.g., a lysophospholipid), or a 54394 protein or polypeptide. Examples of such target molecules include proteins in the same signaling path as the 54394 protein, e.g., proteins which may function upstream (including both stimulators and inhibitors of activity) or downstream of the 54394 protein in a pathway involving regulation of cellular proliferation. Alternatively, a 54394 activity is an indirect activity, such as a cellular signaling activity mediated by interaction of the 54394 protein with a 54394 target molecule. The biological activities of 54394 are described herein. For example, the 54394 proteins can have one or more of the following activities: 1) they modulate hydrolysis of lysophospholipids; 2) they modulate cell membrane integrity; 3) they modulate cell survival, function, and/or lysis; 4) they modulate cellular signaling; and/or 5) they modulate cellular proliferation, growth, apoptosis, differentiation, and/or migration (e.g., in breast, lung, and/or colon cells).

Various aspects of the invention are described in further detail in the following subsections:

I. Screening Assays

The invention provides methods (also referred to herein as “screening assays”) for identifying modulators, i.e., candidate or test compounds or agents (e.g., peptides, peptidomimetics, small molecules, ribozymes, or 54394 antisense molecules) which bind to 54394 proteins, have a stimulatory or inhibitory effect on 54394 expression or 54394 activity, or have a stimulatory or inhibitory effect on the expression or activity of a 54394 target molecule. Compounds identified using the assays described herein may be useful for treating cellular proliferation disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses a 54394 protein or biologically active portion thereof is contacted with a test compound and the ability of the test compound to modulate 54394 activity is determined. In a preferred embodiment, the biologically active portion of the 54394 protein includes a domain or motif which can modulate hydrolysis of a lysophospholipid. Determining the ability of the test compound to modulate 54394 activity can be accomplished by monitoring, for example, the production of one or more specific metabolites (e.g., free fatty acid or other hydrolysis products), by measuring expression of cell cycle regulatory genes, or by monitoring cellular proliferation. The cell, for example, can be of mammalian origin, e.g., a breast cell, a lung cell, or a colon cell.

The ability of the test compound to modulate 54394 binding to a substrate can also be determined. Determining the ability of the test compound to modulate 54394 binding to a substrate (e.g., a lysophospholipid) can be accomplished, for example, by coupling the 54394 substrate with a radioisotope, fluorescent, or enzymatic label such that binding of the 54394 substrate to 54394 can be determined by detecting the labeled 54394 substrate in a complex. Alternatively, 54394 could be coupled with a radioisotope or enzymatic label to monitor the ability of a test compound to modulate 54394 binding to a 54394 substrate in a complex. Determining the ability of the test compound to bind 54394 can be accomplished, for example, by coupling the compound with a radioisotope or enzymatic label such that binding of the compound to 54394 can be determined by detecting the labeled 54394 compound in a complex. For example, 54394 substrates can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with 54394 without the labeling of any of the interactants. For example, a microphysiometer can be used to detect the interaction of a compound with 54394 without the labeling of either the compound or the 54394 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 54394.

Because 54394 expression is increased in tumors, including metastatic tumors, and is regulated during the cell cycle, compounds which modulate cellular proliferation can be identified by the ability to modulate 54394 expression. To determine whether a test compound modulates 54394 expression, a cell which expresses 54394 (e.g., a breast tumor cell, a lung tumor cell, a colon tumor cell, or a corresponding normal cell) is contacted with a test compound, and the ability of the test compound to modulate 54394 expression can be determined by measuring 54394 mRNA by, e.g., Northern Blotting, quantitative PCR (e.g., Taqman), or in vitro transcriptional assays. To perform an in vitro transcriptional assay, the full length promoter and enhancer of 54394 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparing it to reporter gene activity in cells which do not contain the test compound. An increase or decrease in reporter gene activity indicates a modulation of 54394 expression and is, therefore, an indicator of the ability of the test compound to modulate cellular proliferation.

The ability of the test compound to modulate 54394 expression can also be determined by measuring the lysophospholipase activity present in a cell contacted with a test compound. To determine whether a test compound modulates 54394 lysophospholipase activity, a cell which expresses 54394 (e.g., a breast tumor cell, a lung tumor cell, a colon tumor cell, or a corresponding normal cell) is contacted with a test compound, and the ability of the test compound to modulate 54394 lysophospholipase activity can be determined by measuring the intracellular levels of free fatty acid, for example. Exemplary methods for measuring 54394 lysophospholipase activity are described in detail in, for example, Sugimoto, H. et al. (1996) J. Biol. Chem. 271:7705-11; and Wang, A. et al. (1999) Biochim. Biophys. Acta 1437:157-169.

Cell lines transiently and stably transfected with tumor suppressors and oncogenes known to be associated with colon cancer progression may be useful in the methods of the invention for the identification of 54394 modulators (e.g., SW480 cells stably or transiently transfected with Smad4). Smad4 is a candidate tumor suppressor gene mutated in a subset of colon carcinomas. Smad4 functions in the signal transduction of TGF-β molecules. It is well known that the TGF-β superfamily is involved in growth inhibition. Smad4 mutation/loss in colon cell lines provides the hypothesis that Smad4 may be a modulator of cell adhesion and invasion. Other cell lines useful in the methods of the invention are NCM425 cells stably or transiently transfected with β-catenin. Mutations of the APC gene are responsible for tumor formation in sporadic and familial forms of colorectal cancer. APC binds β-catenin and regulates the cytoplasmic levels of β-catenin. When APC is mutated, β-catenin accumulates in the cytoplasm and translocates into the nucleus. Once in the nucleus it interacts with LEF/TCF molecules and regulates gene expression. Genes regulated by the β-catenin/LEF complex, like c-myc and cyclin D1, are involved in tumorigenesis. Also useful in the methods of the invention are cells stably or transiently transfected with p53. p53 is a well known tumor suppressor which is mutated in >50% of colorectal cancer tumors.

Abnormalities in cell cycle regulation and its checkpoints lead to the development of malignant cells. The loss of a cell's ability to respond to signals that regulate cell proliferation and cell cycle arrest is a common mechanism of cancer. Accordingly, for the study of specific time point within the cell cycle, cell lines such as the colon cancer cell lines HCT116, DLD-1 and NCM425 may be synchronized with agents such as Aphidicolin (G1 block), Mimosine (G1 block) and Nocodazole (G2/M block).

Other cell lines useful in the methods of the invention included the colon cancer cell lines HCT116 and DLD1 with disrupted k-ras genes. Point mutations that activate the k-ras oncogene are found in 50% of human colon cancers. Activated k-ras may be regulating cell proliferation in colorectal tumors. Disrupting the activated k-ras allele in HCT116 and DLD1 cells morphologically alters differentiation, causes loss of anchorage independent growth, slows proliferation in vitro and in vivo, and reduces expression of c-myc. Still other cell lines useful in the methods of the invention include transient or stable transfections of WISP-1 into NCM425 colon cancer cells, transient or stable transfections of DCC, Cox2, and/or APC into various cells.

Assays that may be used to identify compounds that modulate 54394 activity also include assays that test for the ability of a compound to modulate cellular proliferation. The ability of a test compound to modulate cellular proliferation can be measured by its ability to modulate proliferation in a cell which expresses 54394, e.g., a breast, lung, or colon cell such as a breast, lung, or colon tumor cell. For example, the ability of a test compound to modulate cellular proliferation can be measured by contacting a cell (e.g., a breast, ovary, lung, or colon tumor cell) with the test compound, incubating the cell for a period of time, and measuring the number of cells present as compared to a control cell not contacted with the test compound. The number of cells can be measured, for example, by dry/wet weight measurement (see Example 1), by counting the cells via optical density (see Example 2), by using a counting chamber (see Example 3), or by using a Coulter Counter. The ability of a test compound to modulate cellular proliferation can also be measured by contacting a cell (e.g., a breast, lung, or colon tumor cell) with the test compound and testing the ability of the cell to form a colony in soft agar (see Example 4). The ability of a cell to grow in soft agar indicates that it has lost the requirement for anchorage-dependant growth, which is an indication of tumorigenic potential. The ability of a test compound to modulate cellular proliferation may also be measured by contacting a cell (e.g., a breast, lung, or colon tumor cell) with the test compound and testing the ability of the cell to form a tumor in a nude mouse. The nude mouse, a hairless mutant discovered in 1962, is immunodeficient, and thus does not reject tumor transplantations from other species.

Numerous other methods exist in the art to measure cellular proliferation. Examples include measurement of the metabolic activity of viable cells via WST-8 reduction to formazan salt using a colorimetric assay (Cell Counting Kit-8 from Alexis Biochemicals, San Diego, Calif. or from Dojindo Molecular Technologies, Inc., Gaithersburg, Md.); measurement of DNA synthesis by BrdU incorporation using an anti-BrdU monoclonal antibody/horseradish peroxidase-based detection system (Cell Proliferation ELISA or Immunocytochemistry from Amersham Pharmacia Biotech, Piscataway, N.J.); DNA synthesis by [¹⁴C]thymidine uptake (Thymidine Uptake [¹⁴C] Cytostar-T Assay from Amersham Pharmacia Biotech, Piscataway, N.J.); and DNA synthesis measured by scintillation proximity assay (SPA) of [³H]thymidine incorporation ([³H]Thymidine Uptake Assay Kit from Amersham Pharmacia Biotech, Piscataway, N.J.).

Further examples of methods for measuring cellular proliferation include measurement of simultaneous cell surface markers and intracellular BrdU incorporation (FastImmune Anti-BrdU with DNase from BD Biosciences, San Jose, Calif.); measurement of the metabolic activity of viable cells via WST-1 reduction to soluble formazan salt using a colorimetric assay (Quick Cell Proliferation Assay Kit from BioVision, Inc., Mountain View, Calif.; Cell Proliferation Assay Kit from Chemicon International, Inc., Temecula, Calif.; Rapid Cell Viability Assay from Oncogene Research Products, San Diego, Calif.; Cell Proliferation Reagent WST-1 from R&D Systems, Minneapolis, Minn.); measurement of live cells stained with “Cyto-dye” and dead cells stained with propidium iodide (Live/Dead Cell Staining Kit from BioVision, Inc., Mountain View, Calif.); and measurement of metabolic activity using bioluminescent detection of ATP (ApoSENSOR ATP Determination Kit from BioVision, Inc., Mountain View, Calif.; LumiTech's ViaLight HS Assay, LumiTech's ViaLight HT Assay, and LumiTech's ViaLight MDA Assay, all from BioWhittaker, Walkersville, Md.; CytoLux Assay Kit from Perkin Elmer Life Sciences, Boston, Mass.; Cytotoxicity and Cell Proliferation Kit from Thermo Labsystems, Franklin, Mass.).

Additional examples of methods for measuring cellular proliferation include measurement of metabolic activity of viable cells via MTT reduction to formazan salt using a colorimetric assay (MTT Cell Growth Assay Kit from Chemicon International, Inc., Temecula, Calif.; Vybrant MTT Cell Proliferation Assay Kit from Molecular Probes, Inc., Eugene, Oreg.; CellTiter 96 Non-Radioactive Cell Proliferation Assay from Promega, Madison, Wis.; TACS MTT Cell Proliferation and Viability Assay and Cell Proliferation Kit I MTT, both from R&D Systems, Minneapolis, Minn.; In Vitro Toxicology Assay Kit, MTT based from Sigma-Aldrich, St. Louis, Mo.); measurement of live cells stained with calcein-AM and dead cells labeled with propidium iodide (Cellstain Double-Staining Kit from Dojindo Molecular Technologies, Inc., Gaithersburg, Md.); measurement of DNA content using CyQUANT GR dye (CyQUANT Cell Proliferation Assay Kit from Molecular Probes, Inc., Eugene, Oreg.); measurement of DNA synthesis by BrdU incorporation using ELISA-based chemiluminescent detection (BrdU Cell Proliferation Assay from Oncogene Research Products, San Diego, Calif.; Cell Proliferation ELISA, BrdU (chemiluminescent) from R&D Systems, Minneapolis, Minn.); and measurement of DNA synthesis by BrdU incorporation using ELISA-based calorimetric detection (BrdU Proliferation Assay—HTS from Oncogene Research Products, San Diego, Calif.; BrdU Labeling and Detection Kit III and Cell Proliferation ELISA, BrdU (calorimetric), both from R&D Systems, Minneapolis, Minn.).

Further examples of methods for measuring cellular proliferation include measurement of proliferating cell nuclear antigen (PCNA) using biotinylated anti-PCNA monoclonal antibody (PCNA (Proliferating Cell Nuclear Antigen) ELISA from Oncogene Research Products, San Diego, Calif.); measurement of DNA synthesis by BrdU incorporation detection using an anti-BrdU monoclonal antibody (BrdU IHC System from Oncogene Research Products, San Diego, Calif.; BrdU Kit from Zymed Laboratories, Inc., South San Francisco, Calif.); measurement of DNA synthesis by BrdU incorporation using Strand Break Induced Photolysis (SBIP) methodology, with break sites identified by BrdU incorporation (ABSOLUTE-S SBIP Cell Proliferation Assay Kit from Phoenix Flow Systems Inc., San Diego, Calif.); measurement of metabolic activity of viable cells via MTS reduction to soluble formazan salt using a colorimetric assay (CellTiter 96 Aqueous Non-Radioactive Cell Proliferation Assay from Promega, Madison, Wis.); and measurement of metabolic activity of viable cells via MTS reduction to formazan salt using a colorimetric assay (CellTiter 96 Aqueous One Solution Cell Proliferation Assay from Promega, Madison, Wis.).

Additional examples of methods for measuring cellular proliferation include measurement of metabolic activity via bioluminescent of ATP using luciferin and thermostable luciferase (CellTiter-Glo Luminescent Cell Viability Assay from Promega, Madison, Wis.); measurement of single-cell proliferation by direct immunofluorescence staining (In Situ Cell Proliferation Kit, FLUOS, and BrdU Labeling and Detection Kit I, both from R&D Systems, Minneapolis, Minn.) or indirect immunostaining method (BrdU Labeling and Detection Kit II from R&D Systems, Minneapolis, Minn.); measurement of metabolic activity of viable cells via XTT reduction to soluble formazan salt using a colorimetric assay (R&D Systems, Minneapolis, Minn.; In Vitro Toxicology Assay Kit, XTT based from Sigma-Aldrich, St. Louis, Mo.); detection of nuclear cell cycle-associated antigens expressed only in proliferating cells (Monoclonal Antibodies to Cell Cycle-Associated Antigens from R&D Systems, Minneapolis, Minn.); measurement of cell proliferation using plasma membrane dye (Cell Census Plus System from Sigma-Aldrich, St. Louis, Mo.); and measurement of membrane-associated phosphatase activity via conversion of p-nitrophenyl phosphate to a colored compound (In Vitro Toxicology Assay Kit, Acid Phosphatase based from Sigma-Aldrich, St. Louis, Mo.).

Still further examples of methods for measuring cellular proliferation include measurement of neutral red dye staining of viable cells using a colorimetric assay (In Vitro Toxicology Assay Kit, Neutral Red based from Sigma-Aldrich, St. Louis, Mo.); measurement of total protein upon sulforhodamine dye binding using a colorimetric assay (In Vitro Toxicology Assay Kit, Sulforhodamine B based from Sigma-Aldrich, St. Louis, Mo.); measurement of metabolic activity of viable cells measured by tetrazolium reduction to formazan derivative using a colorimetric assay (In Vitro Toxicology Assay Kit, Lactic Dehydrogenase based from Sigma-Aldrich, St. Louis, Mo.); measurement of metabolic activity of viable cells via the bioreduction of dye that converts the oxidized form (blue) to a fluorescent intermediate (red) (In Vitro Toxicology Assay Kit, Resazurin based from Sigma-Aldrich, St. Louis, Mo.); measurement of DNA content using Quantos dye reagent (Quantos Cell Proliferation Assay Kit from Stratagene, La Jolla, Calif.); and measurement of DNA content by A:T base pair-binding dye (TACS Hoechst Cell Proliferation Assay I (CPA 1) and TACS Hoechst Cell Proliferation Assay 2 (CPA2), both from Trevigen, Inc., Gaithersburg, Md.).

In yet another embodiment, an assay of the present invention is a cell-free assay in which a 54394 protein or biologically active portion thereof is contacted with a test compound and the ability of the test compound to bind to or to modulate (e.g., stimulate or inhibit) the activity of the 54394 protein or biologically active portion thereof is determined. Preferred biologically active portions of the 54394 proteins to be used in assays of the present invention include fragments which participate in interactions with non-54394 molecules, e.g., fragments with high surface probability scores. Binding of the test compound to the 54394 protein can be determined either directly or indirectly as described above. Determining the ability of the 54394 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a 54394 protein or biologically active portion thereof with a known compound which binds the 54394 protein to form an assay mixture, contacting the assay mixture with a test compound, and determining the ability of the test compound to interact with the 54394 protein, wherein determining the ability of the test compound to interact with the 54394 protein comprises determining the ability of the 54394 protein to preferentially bind to or modulate the activity of a 54394 target molecule (e.g.. a 54394 substrate).

The cell-free assays of the present invention are amenable to use of both soluble and/or membrane-bound forms of isolated proteins (e.g., 54394 proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotriclecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either 54394 or a 54394 target molecule to facilitate separation of complexed from uncomplexed forms of one or both of the proteins, as well as to accommodate automation of the assay. Binding of a test compound to a 54394 protein, or interaction of a 54394 protein with a 54394 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/54394 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 54394 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of 54394 binding or activity determined using standard techniques.

Other techniques for immobilizing proteins or cell membrane preparations on matrices can also be used in the screening assays of the invention. For example, either a 54394 protein or a 54394 target molecule can be immobilized utilizing conjugation of biotin and streptavidin. Biotinylated 54394 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with 54394 protein or target molecules but which do not interfere with binding of the 54394 protein to its target molecule can be derivatized to the wells of the plate, and unbound target or 54394 protein is trapped in the wells by antibody conjugation. Methods for detecting such complexes, in addition to those described above for the GST-immobilized complexes, include immunodetection of complexes using antibodies reactive with the 54394 protein or target molecule, as well as enzyme-linked assays which rely on detecting an enzymatic activity associated with the 54394 protein or target molecule.

In yet another aspect of the invention, the 54394 protein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with 54394 (“54394-binding proteins” or “54394-bp) and are involved in 54394 activity. Such 54394-binding proteins are also likely to be involved in the propagation of signals by the 54394 proteins or 54394 targets as, for example, downstream elements of a 54394-mediated signaling pathway. Alternatively, such 54394-binding proteins are likely to be 54394 inhibitors.

The two-hybrid system is based on the modular nature of most transcription factors, which consist of separable DNA-binding and activation domains. Briefly, the assay utilizes two different DNA constructs. In one construct, the gene that codes for a 54394 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 54394-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 54394 protein.

In another aspect, the invention pertains to a combination of two or more of the assays described herein. For example, a modulating agent can be identified using a cell-based or a cell-free assay, and the ability of the agent to modulate the activity of a 54394 protein can be confirmed in vivo, e.g., in an animal such as an animal model for tumorigenesis, as described elsewhere herein. Additionally, animals deficient in 54394 (e.g., 54394 knockout mice) may be deficient in the ability to modulate cellular proliferation via a 54394-regulated pathway, and therefore may be useful in determining whether a test compound can modulate proliferation by bypassing 54394 and directly modulating the activity of downstream components of the pathway.

This invention further pertains to novel agents identified by the above-described screening assays. Accordingly, it is within the scope of this invention to further use an agent identified as described herein in an appropriate animal model. For example, an agent identified as described herein (e.g., a 54394 modulating agent, an antisense 54394 nucleic acid molecule, a 54394-specific antibody, or a 54394-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

For example, the ability of the agent to modulate the activity of a 54394 protein can be tested in an animal such as an animal model for a cellular proliferation disorder, e.g., tumorigenesis. Animal based models for studying tumorigenesis in vivo are well known in the art (reviewed in Animal Models of Cancer Predisposition Syndromes, Hiai, H. and Hino, O. (eds.) 1999, Progress in Experimental Tumor Research, Vol. 35; Clarke, A. R. (2000) Carcinogenesis 21:435-41) and include, for example, carcinogen-induced tumors (Rithidech, K. et al. (1999) Mutat. Res. 428:33-39; Miller, M. L. et al. (2000) Environ. Mol. Mutagen. 35:319-327), injection and/or transplantation of tumor cells into an animal, as well as animals bearing mutations in growth regulatory genes, for example, oncogenes (e.g., ras) (Arbeit, J. M. et al. (1993) Am. J. Pathol. 142:1187-1197; Sinn, E. et al. (1987) Cell 49:465-475; Thorgeirsson, SS et al. (2000) Toxicol. Lett. 112-113:553-555) and tumor suppressor genes (e.g., p53) (Vooijs, M. et al. (1999) Oncogene 18:5293-5303; Clark A. R. (1995) Cancer Metast. Rev. 14:125-148; Kumar, T. R. et al. (1995) J. Intern. Med. 238:233-238; Donehower, L. A. et al. (1992) Nature 356215-221). Furthermore, experimental model systems are available for the study of, for example, ovarian cancer (Hamilton, T. C. et al. (1984) Semin. Oncol. 11:285-298; Rahman, N. A. et al. (1998) Mol. Cell. Endocrinol. 145:167-174; Beamer, W. G. et al. (1998) Toxicol. Pathol. 26:704-710), gastric cancer (Thompson, J. et al. (2000) Int. J. Cancer 86:863-869; Fodde, R. et al. (1999) Cytogenet. Cell Genet. 86:105-111), breast cancer (Li, M. et al. (2000) Oncogene 19:1010-1019; Green, J. E. et al. (2000) Oncogene 19:1020-1027), melanoma (Satyamoorthy, K. et al. (1999) Cancer Metast. Rev. 18:401-405); lung cancer (Malkinson, A. M. (2001) Lung Cancer 32(3):265-79; Zhao, B. et al. (2001) Exp. Lung Res. 26(8):567-79); colon cancer (Taketo, M. M. and Takaku (2000) Hum. Cell 13(3):85-95; Fodde, R. and Smits, R. (2001) Trends. Mol. Med. 7(8):369-73); and prostate cancer (Shirai, T. et al. (2000) Mutat. Res. 462:219-226; Bostwick, D. G. et al. (2000) Prostate 43:286-294).

Additional examples of mouse models for cancer are detailed below. For example, the Apc^{min} mouse is the most thoroughly characterized genetic model of human colorectal carcinogenesis. This model provides a valuable tool for identifying changes in gene expression associated with early stage disease resulting from the loss of Apc gatekeeper function. Adenomatous polyps and normal colonic epithelium from these mice may be harvested for standard and subtracted cDNA library construction and probe generation for microarray analysis. The Apc^{1638N} mouse was generated by introducing a PGK-neomycin gene at codon 1638 of the Apc gene. After 6-8 weeks, these mice form aberrant crypt foci which ultimately progress to carcinomas by 4 months of age. These mice on average develop 5-6 tumors within the upper gastrointestinal tract. In addition, these mice also develop extraintestinal tumors and desmoids. This lineage provides a means of studying extracolonic manifestations seen in familial adenomatous polyposis (FAP) patients such as desmoid disease. The Smad3^{-/-} mouse has recently been described as a useful and unique model for human colorectal carcinogenesis. Smad3^{-/-} mice develop colon carcinomas that histopathologically resemble human disease. One advantage of this model is that samples from several stages of disease progression can be isolated, including normal epithelium, hyperplastic epithelium, adenomatous polyps, and various degrees of primary carcinoma and lymph node metastases. Thus, the generation of subtracted cDNA libraries and probes representing these stages are a powerful tool for identifying and validating colon cancer targets.

Also useful in the methods of the invention are mis-match repair models (MMRs). Hereditary nonpolyposis colon cancer (HNPCC), which is caused by germline mutations in MSH2 & MLH1, genes involved in DNA mismatch repair, accounts for 5-15% of colon cancer cases. Mouse models have been generated carrying null mutations in the MLH1, MSH2 and MSH3 genes.

Xenograft mouse models are made by grafting cells from colon tumor cell lines into mice, e.g., nude mice. Such genes could be crucial targets for anti-cancer drug development. Examples of colon tumor cell lines which may be used in the methods of the invention to create xenograft mouse models include HCT116, HT29, SW480, SW620, Colon 26, DLD1, Caco2, colo205, T84, CC-ML3, KM12C, KM12SM, HCC-2998, HCT-15, KM20L2, and KM12. Examples of ovary tumor cell lines which may be used in the methods of the invention include cell lines SKOV3, SKOV3/Variant, OVCAR-3, OVCAR-4, and HEY. The SKOV3/Var cell line is a variant of the parental cell line SKOV3 that is resistant to cisplatin.

The HCT-116 human colon carcinoma cell line can be grown as a subcutaneous or orthotopic xenograft (intracaecal injection) in athymic nude mice, but metastasizes with low frequency. Rare liver and lung metastases can be isolated, expanded in vitro, and reimplanted in vivo. A limited (1-3) number of iterations of this process can be employed to isolate highly metastatic variants of the parental cell line. Standard and subtracted cDNA libraries and probes can be generated from the parental and variant cell lines to identify genes associated with the acquisition of a metastatic phenotype. This model can be established using several alternative human colon carcinoma cell lines, including SW480 and KM12C.

Additional animal models which may be useful in the methods of the invention are described in the Examples section herein.

In another aspect, cell-based systems, as described herein, may be used to identify compounds which may act to ameliorate tumorigenic or apoptotic disease symptoms. For example, such cell systems may be exposed to a compound, suspected of exhibiting an ability to ameliorate tumorigenic or apoptotic disease symptoms, at a sufficient concentration and for a time sufficient to elicit such an amelioration of tumorigenic or apoptotic disease symptoms in the exposed cells. After exposure, the cells are examined to determine whether one or more of the tumorigenic or apoptotic disease cellular phenotypes has been altered to resemble a more normal or more wild type, non-tumorigenic disease or non-apoptotic disease phenotype. Cellular phenotypes that are associated with tumorigenic disease states include aberrant proliferation and migration, angiogenesis, anchorage independent growth, and loss of contact inhibition. Cellular phenotypes that are associated with apoptotic disease states include aberrant DNA fragmentation, membrane blebbing, caspase activity, and cytochrome c release from mitochondria.

In addition, animal-based tumorigenic disease systems, such as those described herein, may be used to identify compounds capable of ameliorating tumorigenic or apoptotic disease symptoms. Such animal models may be used as test substrates for the identification of drugs, pharmaceuticals, therapies, and interventions which may be effective in treating tumorigenic or apoptotic disease. For example, animal models may be exposed to a compound, suspected of exhibiting an ability to ameliorate tumorigenic or apoptotic disease symptoms, at a sufficient concentration and for a time sufficient to elicit such an amelioration of tumorigenic or apoptotic tumorigenic or apoptotic disease symptoms in the exposed animals. The response of the animals to the exposure may be monitored by assessing the reversal of disorders associated with tumorigenic disease, for example, by counting the number of tumors and/or measuring their size before and after treatment. In addition, the animals may be monitored by assessing the reversal of disorders associated with tumorigenic disease, for example, reduction in tumor burden, tumor size, and invasive and/or metastatic potential before and after treatment.

With regard to intervention, any treatments which reverse any aspect of tumorigenic or apoptotic disease symptoms should be considered as candidates for human tumorigenic or apoptotic disease therapeutic intervention. Dosages of test agents may be determined by deriving dose-response curves.

Additionally, gene expression patterns may be utilized to assess the ability of a compound to ameliorate cardiovascular or tumorigenic disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, the presence of a tumor, e.g., a breast, colon, or lung tumor, including any of the control or experimental conditions described herein, for example, synchronized cells induced to enter the cell cycle, or RER- or Smad4 models. Other conditions may include, for example, cell differentiation, transformation, metastasis, and carcinogen exposure. Gene expression profiles may be generated, for example, by utilizing a differential display procedure, Northern analysis and/or RT-PCR. In one embodiment, 54394 gene sequences may be used as probes and/or PCR primers for the generation and corroboration of such gene expression profiles.

Gene expression profiles may be characterized for known states, either tumorigenic or apoptotic disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a tumorigenic or apoptotic disease model system to more closely resemble the control system. Administration of a compound may, alternatively, cause the gene expression profile of a control system to begin to mimic a tumorigenic or apoptotic disease state. Such a compound may, for example, be used in further characterizing the compound of interest, or may be used in the generation of additional animal models.

II. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 54394 protein and/or nucleic acid expression as well as 54394 activity, in the context of a biological sample (e.g., blood, serum, cells, or tissue, e.g., breast, lung, or colon tissue) to thereby determine whether an individual is afflicted with a cellular proliferation disorder. The invention also provides for prognostic (or predictive) assays for determining whether an individual is at risk of developing a cellular proliferation disorder. For example, mutations in a 54394 gene can be assayed for in a biological sample. Such assays can be used for prognostic or predictive purpose to thereby prophylactically treat an individual prior to the onset of a cellular proliferation disorder.

Another aspect of the invention pertains to monitoring the influence of 54394 modulators (e.g., anti-54394 antibodies or 54394 ribozymes) on the expression or activity of 54394 in clinical trials.

These and other agents are described in further detail in the following sections.

A. Diagnostic Assays for Cellular Proliferation Disorders

To determine whether a subject is afflicted with a cellular proliferation disorder, a biological sample may be obtained from a subject and the biological sample may be contacted with a compound or an agent capable of detecting a 54394 protein or nucleic acid (e.g., mRNA or genomic DNA) that encodes a 54394 protein, in the biological sample. A preferred agent for detecting 54394 mRNA or genomic DNA is a labeled nucleic acid probe capable of hybridizing to 54394 mRNA or genomic DNA. The nucleic acid probe can be, for example, the 54394 nucleic acid set forth in SEQ ID NO:1, or a portion thereof, such as an oligonucleotide of at least 15, 20, 25, 30, 25, 40, 45, 50, 100, 250 or 500 nucleotides in length and sufficient to specifically hybridize under stringent conditions to 54394 mRNA or genomic DNA. Other suitable probes for use in the diagnostic assays of the invention are described herein.

A preferred agent for detecting 54394 protein in a sample is an antibody capable of binding to 54394 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of direct substances that can be coupled to an antibody or a nucleic acid probe include various enzymes, prosthetic groups, fluorescent materials, luminescent materials, bioluminescent materials, and radioactive materials. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 54394 mRNA, protein, or genomic DNA in a biological sample in vitro as well as in vivo. For example, in vitro techniques for detection of 54394 mRNA include Northern hybridizations and in situ hybridizations. In vitro techniques for detection of 54394 protein include enzyme linked immunosorbent assays (ELISAs), Western blots, immunoprecipitations and immunofluorescence. In vitro techniques for detection of 54394 genomic DNA include Southern hybridizations. Furthermore, in vivo techniques for detection of 54394 protein include introducing into a subject a labeled anti-54394 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 54394 protein, mRNA, or genomic DNA, such that the presence of 54394 protein, mRNA or genomic DNA is detected in the biological sample, and comparing the presence of 54394 protein, mRNA or genomic DNA in the control sample with the presence of 54394 protein, mRNA or genomic DNA in the test sample.

B. Prognostic Assays for Cellular Proliferation Disorder

The present invention further pertains to methods for identifying subjects having or at risk of developing a cellular proliferation disorder with aberrant 54394 expression or activity.

As used herein, the term “aberrant” includes a 54394 expression or activity which deviates from the wild type 54394 expression or activity. Aberrant expression or activity includes increased or decreased expression or activity, as well as expression or activity which does not follow the wild type developmental pattern of expression or the subcellular pattern of expression. For example, aberrant 54394 expression or activity is intended to include the cases in which a mutation in the 54394 gene causes the 54394 gene to be under-expressed or over-expressed and situations in which such mutations result in a non-functional 54394 protein or a protein which does not function in a wild-type fashion, e.g., a protein which does not interact with a 54394 substrate, or one which interacts with a non-54394 substrate.

The assays described herein, such as the preceding diagnostic assays or the following assays, can be used to identify a subject having or at risk of developing a cellular proliferation disorder, e.g., breast cancer, colon cancer, and/or lung cancer. A biological sample may be obtained from a subject and tested for the presence or absence of a genetic alteration. For example, such genetic alterations can be detected by ascertaining the existence of at least one of 1) a deletion of one or more nucleotides from a 54394 gene, 2) an addition of one or more nucleotides to a 54394 gene, 3) a substitution of one or more nucleotides of a 54394 gene, 4) a chromosomal rearrangement of a 54394 gene, 5) an alteration in the level of a messenger RNA transcript of a 54394 gene, 6) aberrant modification of a 54394 gene, such as of the methylation pattern of the genomic DNA, 7) the presence of a non-wild type splicing pattern of a messenger RNA transcript of a 54394 gene, 8) a non-wild type level of a 54394-protein, 9) allelic loss of a 54394 gene, and 10) inappropriate post-translational modification of a 54394-protein.

As described herein, there are a large number of assays known in the art which can be used for detecting genetic alterations in a 54394 gene. For example, a genetic alteration in a 54394 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 54394 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 54394 gene under conditions such that hybridization and amplification of the 54394 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 54394 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 54394 can be identified by hybridizing biological sample derived and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in 54394 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 54394 gene in a biological sample and detect mutations by comparing the sequence of the 54394 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 54394 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 54394 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 54394 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 54394 sequence, e.g., a wild-type 54394 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 54394 genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control 54394 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (199 1) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 54394 modulator (e.g., an agonist, antagonist, peptidomimetic, protein, peptide, nucleic acid, or small molecule) to effectively treat a cellular proliferation disorder.

C. Monitoring of Effects During Clinical Trials

The present invention further provides methods for determining the effectiveness of a 54394 modulator (e.g., a 54394 modulator identified herein) in treating a cellular proliferation disorder in a subject. For example, the effectiveness of a 54394 modulator in increasing 54394 gene expression, protein levels, or in upregulating 54394 activity, can be monitored in clinical trials of subjects exhibiting decreased 54394 gene expression, protein levels, or downregulated 54394 activity. Alternatively, the effectiveness of a 54394 modulator in decreasing 54394 gene expression, protein levels, or in downregulating 54394 activity, can be monitored in clinical trials of subjects exhibiting increased 54394 gene expression, protein levels, or 54394 activity. In such clinical trials, the expression or activity of a 54394 gene, and preferably, other genes that have been implicated in, for example, a cellular proliferation disorder can be used as a “read out” or marker of the phenotype of a particular cell.

For example, and not by way of limitation, genes, including 54394, that are modulated in cells by treatment with an agent which modulates 54394 activity (e.g., identified in a screening assay as described herein) can be identified. Thus, to study the effect of agents which modulate 54394 activity on subjects suffering from a cellular proliferation disorder in, for example, a clinical trial, cells can be isolated and RNA prepared and analyzed for the levels of expression of 54394 and other genes implicated in the cellular proliferation disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 54394 or other genes. In this way, the gene expression pattern can serve as a marker, indicative of the physiological response of the cells to the agent which modulates 54394 activity. This response state may be determined before, and at various points during treatment of the individual with the agent which modulates 54394 activity.

In a preferred embodiment, the present invention provides a method for monitoring the effectiveness of treatment of a subject with an agent which modulates 54394 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 54394 protein, mRNA, or genomic DNA in the pre-administration sample; (iii) obtaining one or more post-administration samples from the subject; (iv) detecting the level of expression or activity of the 54394 protein, mRNA, or genomic DNA in the post-administration samples; (v) comparing the level of expression or activity of the 54394 protein, mRNA, or genomic DNA in the pre-administration sample with the 54394 protein, mRNA, or genomic DNA in the post administration sample or samples; and (vi) altering the administration of the agent to the subject accordingly. For example, increased administration of the agent may be desirable to increase the expression or activity of 54394 to higher levels than detected, i.e., to increase the effectiveness of the agent. Alternatively, decreased administration of the agent may be desirable to decrease expression or activity of 54394 to lower levels than detected, i.e., to decrease the effectiveness of the agent. According to such an embodiment, 54394 expression or activity may be used as an indicator of the effectiveness of an agent, even in the absence of an observable phenotypic response.

III. Methods of Treatment of Subjects Suffering from Cellular Proliferation Disorders

The present invention provides for both prophylactic and therapeutic methods of treating a subject, e.g., a human, at risk of (or susceptible to) a cellular proliferation disorder such as breast cancer, lung cancer, and/or colon cancer. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the 54394 molecules of the present invention or 54394 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a cellular proliferation disorder by administering to the subject an agent which modulates 54394 expression or 54394 activity, e.g., modulation of cellular proliferation in, e.g., breast, lung, or colon cells. Subjects at risk for a cellular proliferation disorder can be identified by, for example, any or a combination of the diagnostic or prognostic assays described herein. Administration of a prophylactic agent can occur prior to the manifestation of symptoms characteristic of aberrant 54394 expression or activity, such that a cellular proliferation disorder is prevented or, alternatively, delayed in its progression. Depending on the type of 54394 aberrancy, for example, a 54394 molecule, 54394 agonist or 54394 antagonist agent can be used for treating the subject. The appropriate agent can be determined based on screening assays described herein.

B. Therapeutic Methods

Another aspect of the invention pertains to methods for treating a subject suffering from a cellular proliferation disorder. These methods involve administering to a subject an agent which modulates 54394 expression or activity (e.g., an agent identified by a screening assay described herein), or a combination of such agents. In another embodiment, the method involves administering to a subject a 54394 protein or nucleic acid molecule as therapy to compensate for reduced, aberrant, or unwanted 54394 expression or activity.

Stimulation of 54394 activity is desirable in situations in which 54394 is abnormally downregulated and/or in which increased 54394 activity is likely to have a beneficial effect, i.e., an increase in cellular proliferation, thereby ameliorating a cellular proliferation disorder such as a neurodegenerative disorder in a subject. Likewise, inhibition of 54394 activity is desirable in situations in which 54394 is abnormally upregulated and/or in which decreased 54394 activity is likely to have a beneficial effect, e.g., an decrease in cellular proliferation, thereby ameliorating a cellular proliferation disorder such as breast cancer, lung cancer, or colon cancer in a subject.

The agents which modulate 54394 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 54394 activity (e.g., a fragment of a 54394 protein or an anti-54394 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 54394 activity can also be prepared in the form of suppositories (e.g., with conventional suppository bases such as cocoa butter and other glycerides) or retention enemas for rectal delivery.

In one embodiment, the agents that modulate 54394 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 54394 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 54394 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al., “Antibodies for Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents in Cancer Therapy: A Review”, in Monoclonal Antibodies '84: Biological and Clinical Applications, Pinchera et al. (eds.), pp. 475-506 (1985); “Analysis, Results, and Future Prospective of the Therapeutic Use of Radiolabeled Antibody in Cancer Therapy”, in Monoclonal Antibodies for Cancer Detection and Therapy, Baldwin et al. (eds.), pp. 303-16 (Academic Press 1985), and Thorpe et al. (1982) “The Preparation and Cytotoxic Properties of Antibody-Toxin Conjugates”, Immunol. Rev. 62:119-58. Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 54394 activity, as well as tailoring the dosage and/or therapeutic regimen of treatment with an agent which modulates 54394 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 54394 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 54394 molecule or 54394 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cellular proliferation disorder with an agent which modulates 54394 activity.

IV. Recombinant Expression Vectors and Host Cells Used in the Methods of the Invention

The methods of the invention (e.g., the screening assays described herein) include the use of vectors, preferably expression vectors, containing a nucleic acid encoding a 54394 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 54394 proteins, mutant forms of 54394 proteins, fusion proteins, and the like).

The recombinant expression vectors to be used in the methods of the invention can be designed for expression of 54394 proteins in prokaryotic or eukaryotic cells. For example, 54394 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 54394 activity assays, (e.g., direct assays or competitive assays described in detail below), or to generate antibodies specific for 54394 proteins. In a preferred embodiment, a 54394 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine box promoters (Kessel and Gruss (1990) Science 249:374-379) and the α-fetoprotein promoter (Campes and Tilghman (1989) Genes Dev. 3:537-546).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 54394 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 54394 nucleic acid molecule of the invention is introduced, e.g., a 54394 nucleic acid molecule within a recombinant expression vector or a 54394 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 54394 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 54394 protein. Accordingly, the invention further provides methods for producing a 54394 protein using the host cells of the invention. In one embodiment, the method comprises culturing the host cell of the invention (into which a recombinant expression vector encoding a 54394 protein has been introduced) in a suitable medium such that a 54394 protein is produced. In another embodiment, the method further comprises isolating a 54394 protein from the medium or the host cell.

V. Isolated Nucleic Acid Molecules Used in the Methods of the Invention

The cDNA sequence of the isolated human 54394 gene and the predicted amino acid sequence of the human 54394 polypeptide are shown in SEQ ID NOs:1 and 2, respectively. Nucleotides 8-718 of SEQ ID NO:1, set forth as SEQ ID NO:3, comprise the 54394 coding region. The human 54394 nucleic acid and amino acid sequences are described in PCT International Publication No. WO98/58066; Hillman et al., U.S. Pat. No. 6,143,544; and in PCT International Publication No. WO01/100806 (amino acid residues 69-237 only); the contents of all of which are incorporated herein by reference.

The methods of the invention include the use of isolated nucleic acid molecules that encode 54394 proteins or biologically active portions thereof, as well as nucleic acid fragments sufficient for use as hybridization probes to identify 54394-encoding nucleic acid molecules (e.g., 54394 mRNA) and fragments for use as PCR primers for the amplification or mutation of 54394 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1 as a hybridization probe, 54394 nucleic acid molecules can be isolated using standard hybridization and cloning techniques (e.g., as described in Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 54394 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO:1, or a portion of any of this nucleotide sequence.

Moreover, the nucleic acid molecules used in the methods of the invention can comprise only a portion of the nucleic acid sequence of SEQ ID NO:1, for example, a fragment which can be used as a probe or primer or a fragment encoding a portion of a 54394 protein, e.g., a biologically active portion of a 54394 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 or an anti-sense sequence of SEQ ID NO:1, or of a naturally occurring allelic variant or mutant of SEQ ID NO:1. In one embodiment, a nucleic acid molecule used in the methods of the present invention comprises a nucleotide sequence which is greater than 50, 100, 100, 150, 200, 250, 300, 350, 400, 450, 500, 550, 600, 650, 700, 750, 800, 850, 900, 950 or more nucleotides in length and hybridizes under stringent hybridization conditions to a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× or 6×sodium chloride/sodium citrate (SSC), at about 65-70° C. (or hybridization in 4×SSC plus 50% formamide at about 42-50° C.) followed by one or more washes in 1×SSC, at about 65-70° C. A further preferred, non-limiting example of stringent hybridization conditions includes hybridization at 6×SSC at 45° C., followed by one or more washes in 0.2×SSC, 0.1% SDS at 65° C. A preferred, non-limiting example of highly stringent hybridization conditions includes hybridization in 1×SSC, at about 65-70° C. (or hybridization in 1'SSC plus 50% formamide at about 42-50° C.) followed by one or more washes in 0.3×SSC, at about 65-70° C. A preferred, non-limiting example of reduced stringency hybridization conditions includes hybridization in 4× or 6×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ (° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 54394 protein, such as by measuring a level of a 54394-encoding nucleic acid in a sample of cells from a subject e.g., detecting 54394 mRNA levels or determining whether a genomic 54394 gene has been mutated or deleted.

The methods of the invention further encompass the use of nucleic acid molecules that differ from the nucleotide sequence shown in SEQ ID NO:1 due to degeneracy of the genetic code and thus encode the same 54394 proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human 54394, e.g., functional and non-functional allelic variants. Functional allelic variants are naturally occurring amino acid sequence variants of the human 54394 protein that maintain a 54394 activity. Functional allelic variants will typically contain only conservative substitution of one or more amino acids of SEQ ID NO:2, or substitution, deletion or insertion of non-critical residues in non-critical regions of the protein. Non-functional allelic variants are naturally occurring amino acid sequence variants of the human 54394 protein that do not have a 54394 activity. Non-functional allelic variants will typically contain a non-conservative substitution, deletion, or insertion or premature truncation of the amino acid sequence of SEQ ID NO:2, or a substitution, insertion or deletion in critical residues or critical regions of the protein.

The methods of the present invention may further use non-human orthologues of the human 54394 protein. Orthologues of the human 54394 protein are proteins that are isolated from non-human organisms and possess the same 54394 activity.

The methods of the present invention further include the use of nucleic acid molecules comprising the nucleotide sequence of SEQ ID NO:1, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 54394 (e.g., the sequence of SEQ ID NO:2) without altering the biological activity, whereas an “essential” amino acid residue is required for biological activity. For example, amino acid residues that are conserved among the 54394 proteins of the present invention and other members of the lysophospholipase family are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 54394 protein is preferably replaced with another amino acid residue from the same side chain family. Alternatively, in another embodiment, mutations can be introduced randomly along all or part of a 54394 coding sequence, such as by saturation mutagenesis, and the resultant mutants can be screened for 54394 biological activity to identify mutants that retain activity. Following mutagenesis of SEQ ID NO:1, the encoded protein can be expressed recombinantly and the activity of the protein can be determined using an assay described herein.

Another aspect of the invention pertains to the use of isolated nucleic acid molecules which are antisense to the nucleotide sequence of SEQ ID NO:1. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 54394 coding strand, or to only a portion thereof. In one embodiment, an antisense nucleic acid molecule is antisense to a “coding region” of the coding strand of a nucleotide sequence encoding a 54394. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 54394. The term “noncoding region” refers to 5′ and 3′ sequences which flank the coding region that are not translated into amino acids (also referred to as 5′ and 3′ untranslated regions).

Given the coding strand sequences encoding 54394 disclosed herein, antisense nucleic acids of the invention can be designed according to the rules of Watson and Crick base pairing. The antisense nucleic acid molecule can be complementary to the entire coding region of 54394 mRNA, but more preferably is an oligonucleotide which is antisense to only a portion of the coding or noncoding region of 54394 mRNA. For example, the antisense oligonucleotide can be complementary to the region surrounding the translation start site of 54394 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl aminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a 54394 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave 54394 mRNA transcripts to thereby inhibit translation of 54394 mRNA. A ribozyme having specificity for a 54394-encoding nucleic acid can be designed based upon the nucleotide sequence of a 54394 cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a 54394-encoding mRNA. See, e.g., Cech et al. U.S. Pat. No. 4,987,071; and Cech et al. U.S. Pat. No. 5,116,742. Alternatively, 54394 mRNA can be used to select a catalytic RNA having a specific ribonuclease activity from a pool of RNA molecules. See, e.g., Bartel, D. and Szostak, J. W. (1993) Science 261:1411-1418.

Alternatively, 54394 gene expression can be inhibited by targeting nucleotide sequences complementary to the regulatory region of the 54394 (e.g., the 54394 promoter and/or enhancers) to form triple helical structures that prevent transcription of the 54394 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In yet another embodiment, the 54394 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. and Nielsen (1996) supra and Perry-O'Keefe et al. (1996) Proc. Natl. Acad. Sci. USA 93:14670-675.

PNAs of 54394 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 54394 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of 54394 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 54394 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (See, e.g., Krol et al. (1988) Biotechniques 6:958-976) or intercalating agents (see, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VI. Isolated 54394 Proteins and Anti-54394 Antibodies Used in the Methods of the Invention

The methods of the invention include the use of isolated 54394 proteins, and biologically active portions thereof, as well as polypeptide fragments suitable for use as immunogens to raise anti-54394 antibodies. In one embodiment, native 54394 proteins can be isolated from cells or tissue sources by an appropriate purification scheme using standard protein purification techniques. In another embodiment, 54394 proteins are produced by recombinant DNA techniques. Alternative to recombinant expression, a 54394 protein or polypeptide can be synthesized chemically using standard peptide synthesis techniques.

As used herein, a “biologically active portion” of a 54394 protein includes a fragment of a 54394 protein having a 54394 activity. Biologically active portions of a 54394 protein include peptides comprising amino acid sequences sufficiently identical to or derived from the amino acid sequence of the 54394 protein, e.g., the amino acid sequence shown in SEQ ID NO:2, which include fewer amino acids than the full length 54394 proteins, and exhibit at least one activity of a 54394 protein. Typically, biologically active portions comprise a domain or motif with at least one activity of the 54394 protein. A biologically active portion of a 54394 protein can be a polypeptide which is, for example, 25, 50, 75, 100, 125, 150, 175, 200, 225 or more amino acids in length. Biologically active portions of a 54394 protein can be used as targets for developing agents which modulate a 54394 activity.

In a preferred embodiment, the 54394 protein used in the methods of the invention has an amino acid sequence shown in SEQ ID NO:2. In other embodiments, the 54394 protein is substantially identical to SEQ ID NO:2, and retains the functional activity of the protein of SEQ ID NO:2, yet differs in amino acid sequence due to natural allelic variation or mutagenesis, as described in detail in subsection V above. Accordingly, in another embodiment, the 54394 protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 54394 amino acid sequence of SEQ ID NO:2 having 237 amino acid residues, at least 71, preferably at least 95, more preferably at least 119, even more preferably at least 142, and even more preferably at least 166, 190, 213 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of Meyers, E. and Miller, W. (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 54394 chimeric or fusion proteins. As used herein, a 54394 “chimeric protein” or “fusion protein” comprises a 54394 polypeptide operatively linked to a non-54394 polypeptide. A “54394 polypeptide” refers to a polypeptide having an amino acid sequence corresponding to a 54394 molecule, whereas a “non-54394 polypeptide” refers to a polypeptide having an amino acid sequence corresponding to a protein which is not substantially homologous to the 54394 protein, e.g., a protein which is different from the 54394 protein and which is derived from the same or a different organism. Within a 54394 fusion protein the 54394 polypeptide can correspond to all or a portion of a 54394 protein. In a preferred embodiment, a 54394 fusion protein comprises at least one biologically active portion of a 54394 protein. In another preferred embodiment, a 54394 fusion protein comprises at least two biologically active portions of a 54394 protein. Within the fusion protein, the term “operatively linked” is intended to indicate that the 54394 polypeptide and the non-54394 polypeptide are fused in-frame to each other. The non-54394 polypeptide can be fused to the N-terminus or C-terminus of the 54394 polypeptide.

For example, in one embodiment, the fusion protein is a GST-54394 fusion protein in which the 54394 sequences are fused to the C-terminus of the GST sequences. Such fusion proteins can facilitate the purification of recombinant 54394.

In another embodiment, this fusion protein is a 54394 protein containing a heterologous signal sequence at its N-terminus. In certain host cells (e.g., mammalian host cells), expression and/or secretion of 54394 can be increased through use of a heterologous signai sequence.

The 54394 fusion proteins used in the methods of the invention can be incorporated into pharmaceutical compositions and administered to a subject in vivo. The 54394 fusion proteins can be used to affect the bioavailability of a 54394 substrate. Use of 54394 fusion proteins may be useful therapeutically for the treatment of disorders caused by, for example, (i) aberrant modification or mutation of a gene encoding a 54394 protein; (ii) mis-regulation of the 54394 gene; and (iii) aberrant post-translational modification of a 54394 protein.

Moreover, the 54394-fusion proteins used in the methods of the invention can be used as immunogens to produce anti-54394 antibodies in a subject, to purify 54394 ligands and in screening assays to identify molecules which inhibit the interaction of 54394 with a 54394 substrate.

Preferably, a 54394 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 54394-encoding nucleic acid can be cloned into such an expression vector such that the fusion moiety is linked in-frame to the 54394 protein.

The present invention also pertains to the use of variants of the 54394 proteins which function as either 54394 agonists (mimetics) or as 54394 antagonists. Variants of the 54394 proteins can be generated by mutagenesis, e.g., discrete point mutation or truncation of a 54394 protein. An agonist of the 54394 proteins can retain substantially the same, or a subset, of the biological activities of the naturally occurring form of a 54394 protein. An antagonist of a 54394 protein can inhibit one or more of the activities of the naturally occurring form of the 54394 protein by, for example, competitively modulating a 54394-mediated activity of a 54394 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 54394 protein.

In one embodiment, variants of a 54394 protein which function as either 54394 agonists (mimetics) or as 54394 antagonists can be identified by screening combinatorial libraries of mutants, e.g., truncation mutants, of a 54394 protein for 54394 protein agonist or antagonist activity. In one embodiment, a variegated library of 54394 variants is generated by combinatorial mutagenesis at the nucleic acid level and is encoded by a variegated gene library. A variegated library of 54394 variants can be produced by, for example, enzymatically ligating a mixture of synthetic oligonucleotides into gene sequences such that a degenerate set of potential 54394 sequences is expressible as individual polypeptides, or alternatively, as a set of larger fusion proteins (e.g., for phage display) containing the set of 54394 sequences therein. There are a variety of methods which can be used to produce libraries of potential 54394 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 54394 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477).

In addition, libraries of fragments of a 54394 protein coding sequence can be used to generate a variegated population of 54394 fragments for screening and subsequent selection of variants of a 54394 protein. In one embodiment, a library of coding sequence fragments can be generated by treating a double stranded PCR fragment of a 54394 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 54394 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 54394 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 54394 variants (Arkin and Youvan (1992) Proc. Natl. Acad. Sci. USA 89:7811-7815; Delagrave et al. (1993) Protein Eng. 6(3):327-331).

The methods of the present invention further include the use of anti-54394 antibodies. An isolated 54394 protein, or a portion or fragment thereof, can be used as an immunogen to generate antibodies that bind 54394 using standard techniques for polyclonal and monoclonal antibody preparation. A full-length 54394 protein can be used or, alternatively, antigenic peptide fragments of 54394 can be used as immunogens. The antigenic peptide of 54394 comprises at least 8 amino acid residues of the amino acid sequence shown in SEQ ID NO:2 and encompasses an epitope of 54394 such that an antibody raised against the peptide forms a specific immune complex with the 54394 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 54394 that are located on the surface of the protein, e.g., hydrophilic regions, as well as regions with high antigenicity.

A 54394 immunogen is typically used to prepare antibodies by immunizing a suitable subject, (e.g., rabbit, goat, mouse, or other mammal) with the immunogen. An appropriate immunogenic preparation can contain, for example, recombinantly expressed 54394 protein or a chemically synthesized 54394 polypeptide. The preparation can further include an adjuvant, such as Freund's complete or incomplete adjuvant, or similar immunostimulatory agent. Immunization of a suitable subject with an immunogenic 54394 preparation induces a polyclonal anti-54394 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 54394. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 54394 molecules. The term “monoclonal antibody” or “monoclonal antibody composition”, as used herein, refers to a population of antibody molecules that contain only one species of an antigen binding site capable of immunoreacting with a particular epitope of 54394. A monoclonal antibody composition thus typically displays a single binding affinity for a particular 54394 protein with which it immunoreacts.

Polyclonal anti-54394 antibodies can be prepared as described above by immunizing a suitable subject with a 54394 immunogen. The anti-54394 antibody titer in the immunized subject can be monitored over time by standard techniques, such as with an enzyme linked immunosorbent assay (ELISA) using immobilized 54394. If desired, the antibody molecules directed against 54394 can be isolated from the mammal (e.g., from the blood) and further purified by well known techniques, such as protein A chromatography to obtain the IgG fraction. At an appropriate time after immunization, e.g., when the anti-54394 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with a 54394 immunogen as described above, and the culture supernatants of the resulting hybridoma cells are screened to identify a hybridoma producing a monoclonal antibody that binds 54394.

Any of the many well known protocols used for fusing lymphocytes and immortalized cell lines can be applied for the purpose of generating an anti-54394 monoclonal antibody (see, e.g., Galfre, G. et al. (1977) Nature 266:55052; Gefter et al. (1977) supra; Lemer (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then (unfused splenocytes die after several days because they are not transformed). Hybridoma cells producing a monoclonal antibody of the invention are detected by screening the hybridoma culture supernatants for antibodies that bind 54394, e.g., using a standard ELISA assay.

Alternative to preparing monoclonal antibody-secreting hybridomas, a monoclonal anti-54394 antibody can be identified and isolated by screening a recombinant combinatorial immunoglobulin library (e.g., an antibody phage display library) with 54394 to thereby isolate immunoglobulin library members that bind 54394. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-54394 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-54394 antibody can be used to detect 54394 protein (e.g., in a cellular lysate or cell supernatant) in order to evaluate the abundance and pattern of expression of the 54394 protein. Anti-54394 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

VII. Electronic Apparatus Readable Media and Arrays

Electronic apparatus readable media comprising 54394 sequence information is also provided. As used herein, “54394 sequence information” refers to any nucleotide and/or amino acid sequence information particular to the 54394 molecules of the present invention, including but not limited to full-length nucleotide and/or amino acid sequences, partial nucleotide and/or amino acid sequences, polymorphic sequences including single nucleotide polymorphisms (SNPs), epitope sequences, and the like. Moreover, information “related to” said 54394 sequence information includes detection of the presence or absence of a sequence (e.g., detection of expression of a sequence, fragment, polymorphism, etc.), determination of the level of a sequence (e.g., detection of a level of expression, for example, a quantitative detection), detection of a reactivity to a sequence (e.g., detection of protein expression and/or levels, for example, using a sequence-specific antibody), and the like. As used herein, “electronic apparatus readable media” refers to any suitable medium for storing, holding, or containing data or information that can be read and accessed directly by an electronic apparatus. Such media can include, but are not limited to: magnetic storage media, such as floppy discs, hard disc storage medium, and magnetic tape; optical storage media such as compact discs; electronic storage media such as RAM, ROM, EPROM, EEPROM and the like; and general hard disks and hybrids of these categories such as magnetic/optical storage media. The medium is adapted or configured for having recorded thereon 54394 sequence information of the present invention.

As used herein, the term “electronic apparatus” is intended to include any suitable computing or processing apparatus or other device configured or adapted for storing data or information. Examples of electronic apparatus suitable for use with the present invention include stand-alone computing apparatuses; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 54394 sequence information. A variety of software programs and formats can be used to store the sequence information on the electronic apparatus readable medium. For example, the sequence information can be represented in a word processing text file, formatted in commercially-available software such as WordPerfect and Microsoft Word, represented in the form of an ASCII file, or stored in a database application, such as DB2, Sybase, Oracle, or the like, as well as in other forms. Any number of dataprocessor structuring formats (e.g., text file or database) may be employed in order to obtain or create a medium having recorded thereon the 54394 sequence information.

By providing 54394 sequence information in readable form, one can routinely access the sequence information for a variety of purposes. For example, one skilled in the art can use the sequence information in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a 54394 associated disease or disorder or a pre-disposition to a 54394 associated disease or disorder, wherein the method comprises the steps of determining 54394 sequence information associated with the subject and based on the 54394 sequence information, determining whether the subject has a 54394 associated disease or disorder or a pre-disposition to a 54394 associated disease or disorder, and/or recommending a particular treatment for the disease, disorder, or pre-disease condition.

The present invention further provides in an electronic system and/or in a network, a method for determining whether a subject has a 54394 associated disease or disorder or a pre-disposition to a disease associated with 54394 wherein the method comprises the steps of determining 54394 sequence information associated with the subject, and based on the 54394 sequence information, determining whether the subject has a 54394 associated disease or disorder or a predisposition to a 54394 associated disease or disorder, and/or recommending a particular treatment for the disease, disorder or pre-disease condition. The method may further comprise the step of receiving phenotypic information associated with the subject and/or acquiring from a network phenotypic information associated with the subject.

The present invention also provides in a network, a method for determining whether a subject has a 54394 associated disease or disorder or a pre-disposition to a 54394 associated disease or disorder associated with 54394, said method comprising the steps of receiving 54394 sequence information from the subject and/or information related thereto, receiving phenotypic information associated with the subject, acquiring information from the network corresponding to 54394 and/or a 54394 associated disease or disorder, and based on one or more of the phenotypic information, the 54394 information (e.g., sequence information and/or information related thereto), and the acquired information, determining whether the subject has a 54394 associated disease or disorder or a pre-disposition to a 54394 associated disease or disorder. The method may further comprise the step of recommending a particular treatment for the disease, disorder or pre-disease condition.

The present invention also provides a business method for determining whether a subject has a 54394 associated disease or disorder or a pre-disposition to a 54394 associated disease or disorder, said method comprising the steps of receiving information related to 54394 (e.g., sequence information and/or information related thereto), receiving phenotypic information associated with the subject, acquiring information from the network related to 54394 and/or related to a 54394 associated disease or disorder, and based on one or more of the phenotypic information, the 54394 information, and the acquired information, determining whether the subject has a 54394 associated disease or disorder or a pre-disposition to a 54394 associated disease or disorder. The method may further comprise the step of recommending a particular treatment for the disease, disorder or pre-disease condition.

The invention also includes an array comprising a 54394 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 54394. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a 54394 associated disease or disorder, progression of 54394 associated disease or disorder, and processes, such a cellular transformation associated with the 54394 associated disease or disorder.

The array is also useful for ascertaining the effect of the expression of a gene on the expression of other genes in the same cell or in different cells (e.g., ascertaining the effect of 54394 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 54394) that could serve as a molecular target for diagnosis or therapeutic intervention.

This invention is further illustrated by the following examples which should not be construed as limiting. The contents of all references, patents and published patent applications cited throughout this application, are incorporated herein by reference.

### EXAMPLES

### Example 1

### Dry/Wet Weight Measurement of Cells

This method of counting cells measures the total weight of cells present in a culture. The cells are grown according to standard protocols in liquid tissue culture. An empty aluminum weighing pan or a sheet of cellulose acetate filter membrane (47 mm in diameter, 0.45 μpore size) is dried in an oven. The pan or filter is then weighed and stored in a desiccator lined with Drierite (anhydrous CaSO4). The culture flask is stirred to suspend the culture evenly. 100 ml of the culture is poured into a graduated cylinder. The cells are separated from the medium either by centrifugation at 10,000 g for 5 minutes or by filtration. In the case of centrifugation, the supernatant is discarded, and the pelleted cell paste is scraped from the centrifuge tube into a weighing pan. The centrifuge tube is then rinsed with a few ml of water, and the rinse water is poured into the weighing pan as well. In the case of filtration, the culture is poured into the holding reservoir fitted on the filter membrane. A vacuum is then applied to pull the liquid through the membrane. The reservoir is rinsed with a few ml of water, and any cell paste adhering to the glassware are scraped off. The wet weight of the culture is measured immediately after all the water has been pulled through the filter.

The cell paste is placed in an oven set at 100° C. The cells will be charred and the filter membrane will be burned if the temperature of the oven is set too high. The weight of the pan/filter plus the cell paste is weighed periodically until there is no further decrease in the dry weight. It will take 6-24 hours to dry the sample completely, depending on the oven temperature and the thickness of the paste. The difference in the weight is calculated, and the dry weight is expressed in g/l.

### Example 2

### Measurement of Cells Via Optical Density

The cells are grown according to standard protocols in liquid tissue culture. The culture sample is diluted to appropriate concentrations as needed, and the absorbance of the sample is measured with a spectrophotometer at 550 nm. Other wavelengths may also be used. A calibration curve is generated to correlate the absorbance with cell dry weight. The accuracy of the method is the highest when the absorbance is between 0.1 and 0.5.

For a given culture sample, a good spectrophotometer should yield a linear relationship between the number of cells and the absorbance. However, the optical density is also a function of cell morphology such as size and shape, because the amount of transmitted or scattered light depends strongly on these factors. Consequently, an independent calibration curve is required for each condition, as the cell size and shape depend on the specific growth rate and the nutrient composition. Generally, an optical density of 1 unit corresponds to approximately 1 g/l of dry cell.

### Example 3

### Measurement of Cells Using a Cell Counting Chamber

The cells are grown according to standard protocols in liquid tissue culture. The most widely used type of chamber is called a hemocytometer, since it was originally designed for performing blood cell counts. The counting chamber is prepared by carefully cleaning the mirror-like polished surface with lens paper. The coverslip is also cleaned. Coverslips for counting chambers are specially made and are thicker than those for conventional microscopy, since they must be heavy enough to overcome the surface tension of a drop of liquid. The coverslip is placed over the counting surface prior to putting on the cell suspension. The suspension is introduced into one of the V-shaped wells with a pasteur or other type of pipet. The area under the coverslip fills by capillary action. Enough liquid should be introduced so that the mirrored surface is just covered. The charged counting chamber is then placed on a microscope stage and the counting grid is brought into focus at low power.

It is essential to be extremely careful with higher power objectives, since the counting chamber is much thicker than a conventional slide. The chamber or an objective lens may be damaged if the user is not careful. One entire grid on standard hemocytometers with Neubauer rulings can be seen at 40× (4×objective). The main divisions separate the grid into 9 large squares (like a tic-tac-toe grid). Each square has a surface area of one square mm, and the depth of the chamber is 0.1 mm. Thus, the entire counting grid lies under a volume of 0.9 mm-cubed.

Cell suspensions should be dilute enough so that the cells do not overlap each other on the grid, and should be uniformly distributed. To perform the count, the magnification needed to recognize the desired cell type is determined. The cells are then systematically counted in selected squares so that the total count is 100 cells or so (number of cells needed for a statistically significant count). For large cells, this may mean counting the four large corner squares and the middle one. For a dense suspension of small cells, it may be desirably to count the cells in the four 1/25 sq. mm corners plus the middle square in the central square. A specific counting pattern is used to avoid bias. For cells that overlap a ruling, the cell is counted as “in” if it overlaps the top or right ruling, and “out” if it overlaps the bottom or left ruling.

To determine a cell count using a standard hemocytometer, the total count is divided by 0.1 (chamber depth), and the result is divided by the total surface area counted. For example, if 125 cells were counted in each of the four large corner squares plus the middle, 125 is divided by 0.1, and the result is divided by 5 mm-squared, which is the total area counted (each large square is 1 mm-squared). 125/0.1=1250. 1250/5=250 cells/mm-cubed. There are 1000 mm-cubed per ml, so there are therefore 250,000 cells/ml. Sometimes the cell suspension will need to be diluted to get the cell density low enough for counting. In that case, the final count is divided by the dilution factor.

### Example 4

### Soft Agar Assay for Anchorage-Independent Growth of Cells

Base Agar

1% Agar (DNA grade) is melted in a microwave and cooled to 40° C. in a waterbath. 2×RPMI medium+20% fetal calf serum (FCS) is also warmed to 40° C. in a waterbath. Equal volumes of the two solutions are mixed to give 0.5% Agar+1×RPMI+10% FCS. 1.5 ml is poured into each 35 mm Petri plate and allowed to set. The plates can be stored at 4° C. for up to 1 week.

Top Agar

0.7% Agarose (DNA grade) is melted in a microwave and cooled to 40° C. in a waterbath. 2×RPMI+20% FCS is also warmed to the same temperature.

Cells

The cells (e.g., breast, lung, or colon cells) to be assayed are trypsinized, suspended in medium, and counted. A positive control, such as a ras transformed cell line, should always be used. The concentration of the cell suspension is adjusted to 200,000 cells/ml.

Plating and Staining

0.1 ml of cell suspension is added to 10 ml capped centrifuge tubes. The 35 mm Petri plates containing the base agar are removed from 4° C. about 30 minutes prior to plating to allow them to warm up to room temperature. 3ml 2×RPMI+10% or 20% FCS and 3 ml 0.7% Agarose are added to each tube of cell suspension and mixed gently. 1.5 ml of this mixture is added to each replicate plate (each plate is done in triplicate), and the agarose is allowed to solidify. The plates are incubated at 37° C. in humidified incubator for 10-14 days. After completion of the incubation period, the plates are stained with 0.5 ml of 0.005% Crystal Violet for at least 1 hour. The colonies are then counted using a dissecting microscope.

### Example 5

### Tissue Expression Analysis of Human 54394 mRNA Using Taqman Analysis

This example describes the tissue distribution of human 54394 mRNA, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest and used as the starting material for PCR amplification. In addition to the 5′ and 3′ gene-specific primers a gene-specific oligonucleotide probe (complementary to the region being amplified) was included in the reaction (i.e., the Taqman™ probe). The TaqMan™ probe included the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separated the reporter dye and the quencher dye, resulting in increased fluorescence of the reporter. Accumulation of PCR products was detected directly by monitoring the increase in fluorescence of the reporter dye. When the probe was intact, the proximity of the reporter dye to the quencher dye resulted in suppression of the reporter fluorescence. During PCR, if the target of interest is present, the probe specifically annealed between the forward and reverse primer sites. The 5′-3′ nucleolytic activity of the AmpliTaq™ Gold DNA Polymerase cleaved the probe between the reporter and the quencher only if the probe hybridized to the target. The probe fragments were then displaced from the target, and polymerization of the strand continued. The 3′ end of the probe was blocked to prevent extension of the probe during PCR. This process occurred in every cycle and did not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

For in situ analysis, various tumors and normal tissues were first frozen on dry ice. Ten-micrometer-thick sections of the tissues were postfixed with 4% formaldehyde in DEPC-treated 1×phosphate-buffered saline at room temperature for 10 minutes before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15 M NaCl plus 0.015 M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections were then sequentially incubated at 37° C. in TNE (a solution containing 10 mM Tris-HCl (pH 7.6), 500 mM NaCl, and 1 mM EDTA1), for 10 minutes, in TNE with 10 μg of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

The expression of human 54394 mRNA was examined in various cell types and tissues using Taqman analysis. As shown in Table 1, 54394 is highly expressed in normal artery, diseased aorta, normal vein, coronary smooth muscle cells, human umbilical vein endothelial cells, hemangioma, normal heart, heart under conditions of congestive heart failure, kidney, skeletal muscle, pancreas, normal brain cortex, hypothalamus, megakaryocytes, and erythroid cells.

The expression of human 54394 was also examined in different tumors using Taqman analysis. As shown in Table 2, 54394 is upregulated in 1/5 ovary tumors, as compared to normal ovary tissue; in 3/5 lung tumors, as compared to normal lung tissue; in 2/3 colon tumors, as compared to normal colon tissue. The colon tumors with upregulated expression showed a 40-100 fold higher expression level than the normal colon tissue samples. The results in Table 2 also indicate that 54394 expression is downregulated in HCT116 colon cancer cells under hypoxic conditions, as compared to HCT116 cells under normoxic conditions.

The expression of human 54394 was also examined in a number of xenograft friendly colon tumor cell lines using Taqman analysis. These cell lines can produce tumors when injected into mice. As shown in Table 3, 54394 is highly expressed in MCF-7 breast tumor cells, ZR75 breast tumor cells, T47D breast tumor cells, DLD1 (stage C) colon cancer cells, SW620 (stage C) colon cancer cells, NCIH67 lung cancer cells, and NCIH460 lung cancer cells.

The expression human 54394 was also examined in various solid human colon tumors at different stages of tumorigenesis using Taqman analysis. As shown in Table 4, 54394 is upregulated in 4/5 metastases to the liver, as compared to normal colon tissue. These samples do not show upregulation, however, when compared with normal liver. Further examination of 54394 expression in metastases using Taqman analysis did not show upregulation (Table 5). However, transcriptional profiling analysis showed that 54394 expression was upregulated in 15/15 colon metastases to the liver.

In situ hybridization analysis indicated that human 54394 was expressed in polyps (1/1 positive sample), tumors (3/5 positive samples) metastatic carcinomas (6/6 positive samples), and normal liver (2/2 positive samples), but not in normal colon (0/4 samples). In situ hybridization analysis also indicated that human 54394 was expressed in 3/6 lung

The expression of human 54394 was further examined in HCT116 cell lines with disrupted k-ras genes. As shown in Table 6, 54394 expression is downregulated when k-ras is inactivated in HCT116 cell lines.

The expression of human 54394 was further examined in synchronized tumor cells induced to enter the cell cycle. As shown in Table 7, expression of 54394 was not regulated in HCT 116 colon cancer cells synchronized with Nocodazole, which blocks at the G2/M stage of the cell cycle. Expression of 54394 was also regulated in A549 lung tumor cells synchronized with Nocodazole.

The expression of human 54394 was further examined in in vitro colon cancer models. The results are set forth in Tables 8-10, which show many of the same samples examined in separate experiments. The results indicate that 54394 is upregulated in SW480 cells that have been transfected with SMAD4. 54394 is also expressed at lower levels in non-mucinous tumor cells than in mucinous cells. 54394 is highly expressed in SW48 RER+ cells, JDLD-1 cells, JHCT116 cells, DKO1 cells, DKO4 cells, DKS-8 cells, HK2-6 cells, and in e3Ham#9 cells.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating a cell proliferation disorder, comprising assaying the ability of the compound to modulate 54394 nucleic acid expression or 54394 polypeptide activity, thereby identifying a compound capable of treating a cell proliferation disorder.

2. A method for identifying a compound capable of modulating cellular proliferation comprising:
a) contacting a cell which expresses 54394 with a test compound;and
b) assaying the ability of the test compound to modulate the expression of a 54394 nucleic acid or the activity of a 54394 polypeptide, thereby identifying a compound capable of modulating cellular proliferation.

3. A method for modulating cellular proliferation in a cell comprising contacting a cell with a 54394 modulator, thereby modulating cellular proliferation in the cell.

4. The method of claim 2, wherein the cell is a breast cell, a lung cell or a colon cell.

5. The method of claim 3, wherein the 54394 modulator is a small organic molecule, peptide, antibody or antisense nucleic acid molecule.

6. The method of claim 3, wherein the 54394 modulator is capable of modulating 54394 polypeptide activity.

7. The method of claim 6, wherein the 54394 modulator is a small organic molecule, peptide, antibody or antisense nucleic acid molecule.

8. The method of claim 6, wherein the 54394 modulator is capable of modulating 54394 nucleic acid expression.

9. A method for treating a subject having a cell proliferation disorder characterized by aberrant 54394 polypeptide activity or aberrant 54394 nucleic acid expression comprising administering to the subject a 54394 modulator, thereby treating said subject having a cell proliferation disorder.

10. The method of claim 9, wherein said cell proliferation disorder is selected from the group consisting of breast cancer, lung cancer and colon cancer.

11. The method of claim 9, wherein said 54394 modulator is administered in a pharmaceutically acceptable formulation.

12. The method of claim 9, wherein the 54394 modulator is a small organic molecule, peptide, antibody or antisense nucleic acid molecule.

13. The method of claim 9, wherein the 54394 modulator is capable of modulating 54394 polypeptide activity.


SEQUENCE LISTING (3 sequences)

SEQ ID NO: 1 - DNA, 975 bp, Homo sapien
atcagcgatggcggctgcgtcggggtcggttctgcagcgctgtatcgtgtcgccggcagggaggcatagcgcctctctgatcttcctgcatggctcaggtgattctggacaaggattaagaatgtggatcaagcaggttttaaatcaagatttaacattccaacacataaaaattatttatccaacagctcctcccagatcatatactcctatgaaaggaggaatctccaatgtatggtttgacagatttaaaataaccaatgactgcccagaacaccttgaatcaattgatgtcatgtgtcaagtgcttactgatttgattgatgaagaagtaaaaagtggcatcaagaagaacaggatattaataggaggattctctatgggaggatgcatggcaatgcatttagcatatagaaatcatcaagatgtggcaggagtatttgctctttctagttttctgaataaagcatctgctgtttaccaggctcttcagaagagtaatggtgtacttcctgaattatttcagtgtcatggtactgcagatgagttagttcttcattcttgggcagaagagacaaactcaatgttaaaatctctaggagtgaccacgaagtttcatagttttccaaatgtttaccatgagctaagcaaaactgagttagacatattgaagttatggattcttacaaagctgccaggagaaatggaaaaacaaaaatgaatgaatcaagagtgatttgttaatgtaagtgtaatgtctttgtgaaaagtgatttttactgccaaattataatgataattaaaatattaagaaataacactttcctgacttttttattattaaaatgcttatcactgtagacagtagctaatcttattaatgaaaaacaatagacaaacatctgtgcataatttttcagacacaattctgtaaatatttggaaaccttttaagtatttaaacttttaaattttt

SEQ ID NO: 2 - protein, 237 aa, Homo sapien
MAAASGSVLQRCIVSPAGRHSASLIFLHGSGDSGQGLRMWIKQVLNQDLTFQHIKIIYPTAPPRSYTPMKGGISNVWFDRFKITNDCPEHLESIDVMCQVLTDLIDEEVKSGIKKNRILIGGFSMGGCMAMHLAYRNHQDVAGVFALSSFLNKASAVYQALQKSNGVLPELFQCHGTADELVLHSWAEETNSMLKSLGVTTKFHSFPNVYHELSKTELDILKLWILTKLPGEMEKQK

SEQ ID NO: 3 - DNA, 975 bp, Homo sapien; identical to SEQ ID NO: 1
  CDS (8)...(721)